(19) United States
(12) Patent Application Publication
(10) Pub. No.: US 2006/0083722 A1
(43) Pub. Date: Apr. 20, 2006

(54) Methods and compositions for cell therapy

(75) Inventors: Jose Cibelli, East Lansing, MA (US); Tanja Dominko, Southbridge, MA (US)

Correspondence Address: FISH & NEAVE IP GROUP;ROPES & GRAY LLP, 1251 AVENUE OF THE AMERICAS FL C3, NEW YORK, NY, 10020-1105, US

(73) Assignee: Advanced Cell Technology, Inc, Worcester, MA (US)

(21) Appl. No.: 10/484,398

(22) PCT Filed: Jan. 25, 2002
(86) PCT No.: PCT/US02/02285

§ 371 Date: Dec. 9, 2005

(30) Foreign Application Priority Data
Jul. 18, 2001 (US) 60305904

(51) Int. Cl. A61K 48/00; C12N 5/08

(52) U.S. Cl. 424/93.21; 435/366

(57) ABSTRACT

Improved methods of cell therapy are provided using cells and tissues that are histocompatible with a human or non-human mammal transplant recipient. The cells and tissues for transplant produced by the present invention exhibit a youthful state and can be committed to specific cell lineages to better infiltrate and proliferate at a desired target, e.g., a tissue, or organ in need of cell replacement therapy. For providing cells and tissues for transplant to a non-human mammal, the cells and tissues can be isolated from a gastrulating embryo produced by same-species nuclear transfer. Histocompatible cells and tissues for transplant to a human can be isolated from a gastrulating embryo that (i) is genetically modified to be in capable of developing beyond and early stage, or (ii) is produced by cross-species nuclear transfer between a human nuclear donor cell and an enucleated recipient cell, e.g., an oocyte, of a non-human mammal, or (iii) is produced by androgenesis or gynogenesis, or from pluripotent stem cells generated from such an embryo. Methods for producing histocompatible cells and tissues for transplant to a human can also be used to produce such cells or tissues for transplant to non-human mammals. The present invention also provides model embryonic systems having defined genetic makeup that are useful for developing and testing methods for cell and tissue therapy, and for studying genetic imprinting, reprogramming, rejuvenation, and other biochemical, metabolic, and physiological phenomena associated with embryogenesis.

### FIELD OF THE INVENTION

The present invention relates to novel and improved methods and compositions for cell and tissue therapy. The invention relates to methods for producing cell and tissue compositions suitable for therapeutic transplantation to a mammal in need of a therapeutic cell or tissue transplant. The invention relates to methods for producing cell and tissue compositions suitable for therapeutic transplantation that are histocompatible with an individual mammal in need of such a cell or tissue transplant. The invention relates to producing such histocompatible cell and tissue compositions for transplant by methods comprising somatic cell nuclear transfer and/or androgenesis or gynogenesis. The invention further relates to methods for producing and using model embryonic, fetal, and developed animal systems having defined genetic makeup that are of use in developing and testing methods for cell and tissue therapy, and as model systems for studying imprinting, reprogramming, rejuvenation, and other biochemical, metabolic, and physiological phenomena associated with embryogenesis and development.

### BACKGROUND

Great Need for Histocompatible Cells and Tissues for Transplant

There presently is great need for new sources of cells and tissues for therapeutic transplant that are histocompatible with the transplant recipients. Transplanted cells or tissue are rejected by the immune system of the transplant recipient unless they are histocompatible with the recipient. Rejection occurs as a result of an adaptive immune response to alloantigens on the grafted tissue by the transplant recipient. The alloantigens are “non-self” proteins, i.e., antigenic proteins that are identified as foreign by the immune system of a transplant recipient. Recognition of foreign antigens on the transplant by the recipient's T cells sets in motion a chain of signaling and regulatory events that causes the activation and recruitment of additional T cells and other cytotoxic cells, and culminates in the destruction of the transplanted tissue. The proteins on the surfaces of transplanted tissue that most strongly evoke rejection are the antigenic MHC proteins. Assays are used to identify the MHC types present on the cells of tissue to be transplanted and on the cells of transplant recipients, in order to match the types of MHC molecules present in the transplant tissue with those of the recipient. Matching the MHC molecules of a transplant to those of the recipient significantly improves the success rate of clinical transplantation; however, it does not prevent rejection, even when the transplant is between HLA-identical siblings. This is because rejection is also triggered by differences between the minor histocompatibility antigens—polymorphic, antigenic “non-self” peptides that are bound to MHC molecules on the cells of the transplant tissue. The rejection response evoked by a single minor histocompatibility antigen is much weaker than that evoked by differences in MHC antigens, because the frequency of the responding T cells is much lower. Nonetheless, differences between minor histocompatibility antigens will cause the immune system of a transplant recipient to eventually reject a transplant, even where there is a match between the MHC antigens, unless immunosuppressive drugs are used. The number of people in need of cell, tissue, and organ transplants is far greater than the available supply of cells, tissues, and organs suitable for transplantation. As a result, it is frequently impossible to obtain a good match between a recipient's MHC proteins those of cells or tissue that are available for transplant. Hence, many transplant recipients must wait for an MHC-matched transplant to become available, or accept a transplant that is not MHC-matched. If the latter is necessary, the transplant recipient must rely on heavier doses of immunosuppressive drugs and face a greater risk of rejection than would be the case if MHC matching had been possible. New sources of histocompatible cells and tissues for therapeutic transplant to non-human mammals in need of such transplant also will be of great value in veterinary medicine.

Histocompatible Cells and Tissues Produced by Nuclear Transfer into Oocytes

Cloning methods employing the technique of nuclear transfer have been developed and used widely in recent years to produce clones of valued mammals of a variety of species, including cattle, pigs, sheep, goats, and cats. Cloning by nuclear transfer comprises transferring the nucleus of a cell of a mammal to be cloned into an oocyte from which the maternal DNA is removed. Such methods are of great value in agriculture, as they allow for production of an essentially limitless supply of cloned animals having desirable characteristics, e.g., size, fat/muscle ratio, immunity and resistance to disease, etc. The production of cloned animals by nuclear transfer has additional utility because it provides an efficient means for producing cloned transgenic animals. Cells isolated from an animal to be cloned can be genetically modified in vitro by introduction of desired heterologous DNA sequences; e.g., DNA sequences that encode proteins that have therapeutic activity, industrial utility, or other commercial value, or that prevent the expression of one or more genes. Cloned transgenic animals that have the genomic DNA of the genetically modified donor cells and express the heterologous DNA sequences in one or more tissues can then be produced by using the genetically modified cells used as donor cells in cloning by nuclear transfer.

Cloning by nuclear transfer can also be used to produce cells and tissues for therapeutic transplantation to humans or animals individuals in need of such treatment. When a cell from the individual in need of transplant therapy is used as the donor cell, nuclear transfer cloning produces an embryo having the same genomic DNA as the transplant recipient. As a result, the cells and tissues generated from such an embryo are nearly completely autologous—all of the cells' proteins except those encoded by the cells' mitochondria, which de rive from the oocyte, are encoded by the patient's own DNA. Hence, these cells and tissues can be used for transplantation without triggering the severe rejection response that results when foreign cells or tissue are transplanted.

Advanced Cell Technology, Inc. (ACT), the assignee of this application, has shown that nuclear transfer cloning can generate embryos that are “hyper-youthful”—their cells have longer telomeres and a longer proliferative life-span that those of age-matched control cells of the same type and species that are not generated by nuclear transfer techniques. Researchers at ACT have also shown that the immune systems of cloned animals produced by nuclear transfer procedures are enhanced, i.e., show greater immune response, relative to those of animals that are not generated by nuclear transfer techniques.

Cells and tissues suitable for therapeutic transplantation to humans or animals can be obtained directly from a fetus grown from a nuclear transfer embryo; alternatively, a nuclear transfer embryo can be cultured in vitro to generate pluripotent embryonic stem cells, and these can be cultured and induced to differentiate into various kinds of stem cells, cell lineages, and differentiated cell types for transplant. According to data from the Centers for Disease Control and Prevention), as many as 3,000 Americans die every day from diseases that in the future may be treatable with tissues derived from embryonic stem (ES) cells. In addition to generating functional replacement cells such as cardiomyocytes, neurons, or insulin-producing β cells, ES cells may be able to reconstitute more complex tissues and organs, including blood vessels, myocardial “patches,” kidneys, and even entire hearts²⁻⁴. Somatic cell nuclear transfer has the potential to eliminate immune responses associated with the transplantation of such tissues and thus the requirement for immunosuppressive drugs and/or immunomodulatory protocols, which carry the risk of serious and potentially life-threatening complications⁵.

Methods for producing histocompatible cells and tissues suitable for transplant that involve destruction of a viable nuclear transfer embryo are acceptable when the embryo is that of a non-human animal; however, alternative procedures must be followed when the donor cell used in nuclear transfer cloning is that of a human. One approach for producing histocompatible, syngenic cells and tissues for a human transplant recipient is to genetically modify the donor cell so that it gives rise to an embryo that is incapable of developing beyond an early stage of embryonic development. Another approach is to transfer the human donor cell into an oocyte of a non-human mammal to produce an embryo that cannot develop into a human being. There is thus a need for new and improved methods employing nuclear transfer cloning to provide cells and tissues suitable for transplant for humans and to non-human animals.

Cells from an Nuclear Transplant Embryo are not Rejected by a Syngenic Transplant Recipient

Recent studies by researchers at ACT have shown that cells and tissues isolated from an embryo produced by nuclear transfer cloning and transplanted into syngenic cattle do not elicit rejection. For example, Lanza et al. report that tissue-engineered constructs comprising three different differentiated cell types isolated from a bovine nuclear transplant embryo were transplanted into syngenic cattle, where they survived and grew for 12 weeks without rejection, while allogenic control cells were rejected (see Nature Biotechnology, 2002, 20:689-695, the contents of which are incorporated herein in their entirety). Lanza et al. further demonstrated that the nucleotide sequence of the mitochondrial DNA of the unrejected transplant cells was not the same as the sequence of the mitochondrial DNA transplant recipient, and encoded expressed proteins that are structurally different from those produced by the mitochondria of the transplant recipient. These results are included in Example 3. This work helps to allay fears that allogenic mitochondria in cells and tissues obtained from a nuclear transfer embryo and transplanted into a syngenic transplant recipient would elicit rejection of the transplant because the immune system of the transplant recipient would detect foreign proteins encoded by the allogenic mitochondrial DNA in the transplanted cells.

Cells and Tissues for Transplant from Androgenetic and Gynogenetic Embryos

Histocompatible cells and tissues suitable for transplant to humans can also be generated from nonviable gynogenetic or androgenetic embryos that are produced to have the genomic DNA of a female or male transplant recipient.

Under certain conditions that may occur spontaneously or by design in vivo or in vitro, oocytes containing genomic DNA of all-male or all-female origin may become activated and produce a zygote or zygote-like cell that can undergo cleavage and subsequent mitotic division. Gynogenesis is broadly defined as the phenomena wherein an oocyte containing all-female DNA becomes activated and produces an embryo. Gynogenesis includes the production of an embryo having all-female genomic DNA by a process in which the oocyte is activated to complete meiosis by a sperm cell that fails to contribute any genetic material to the resulting embryo. Parthenogenesis is a type of gynogenesis in which an oocyte containing all-female genomic DNA is activated to produce an embryo without any interaction with a male gamete. Parthenogenetically activated oocytes may experience aberrations during the completion of meiosis that result in the production of embryos of aberrant genetic constitutions; e.g., embryos that are polyploid or mixoploid. Androgenesis is in many respects the opposite of gynogenesis; it is a phenomenon whereby an oocyte containing genomic DNA exclusively of male origin is produced and activated to develop into an embryo having all-male genomic DNA. Both haploid and diploid gynogenetic and androgenetic embryos may be produced. Gynogenetic and androgenetic embryos typically stop developing at a fairly early stage in embryogenesis, because the maternal and paternal chromosomes are structurally and functionally different from each other, and both types of chromosomes are generally needed for normal embryonic development to proceed. There is thus a need for new, improved methods for producing gynogenetic and androgenetic embryos from which can be generated cells and tissues that are suitable for transplant to humans and non-human mammals.

Imprinting and Epigenetic Chromosomal Modifications

Genes that are present on both the maternal and paternal chromosomes, but which are differentially expressed, depending on whether they are located on the maternal or the paternal chromosome, are referred to as being imprinted. An example of an imprinted gene is the Igf2 gene that is located on the chromosome 7 and encodes insulin-like growth factor II (IGFII), a potent embryonic mitogen. The Igf2 gene on the paternal copy of chromosome 7 is actively expressed in embryonic cells, whereas the maternal copy of chromosome 7 is inactive. The differential expression of imprinted genes in embryonic cells is due to epigenetic structural differences between the maternal and paternal chromosomes; i.e., to structural modifications that do not result in differences in the nucleotide sequences of the genes present on the maternal and paternal chromosomes. Patterns of gene expression are also affected by genomic imprinting in cells of adult mammals. Syndromes and diseases in humans associated with genomic imprinting include Prader-Willi syndrome, Angelman syndrome, uniparental isodisomy, Beckwith-Wiedermann syndrome, Wilm's tumor carcinogenesis and von Hippel-Lindau disease. In animals, genomic imprinting has been linked to coat color. For example, the mouse agouti gene confers wild-type coat color, and differential expression of the Aiapy allele correlates with the methylation status of the gene's upstream regulatory sequences. There currently is great interest in identifying how chromosomes contributed to the embryo by male gametes are structurally and functionally different from the chromosomes contributed to female gametes, e.g., in the regulation of differential expression of imprinted genes, and the role these epigenetic differences play in the development of the embryo. Hence, there is a need for methods for producing haploid and diploid androgenetic and gynogenetic embryos that are useful as model systems for studying the epigenetic structural differences between the chromosomes of sperm and egg, and their role in embryogenesis.

### BRIEF DESCRIPTION OF THE DRAWINGS

FIG. 1 shows a parthenogenetically activated rabbit blastocyst at day 8 (scale bar=100 microns).

FIG. 2 shows a parthenogenetically activated rabbit blastocyst/embryonic sac cultured in vitro at day 22 (scale bar=500 microns).

FIG. 3 shows embryonic cells isolated from parthenogenetically activated rabbit blastocyst/embryonic sac at day 22 (scale bar=50 microns).

FIG. 4—Retrieved muscle tissues. (A) Retrieved cloned cardiac tissue shows a well-organized cellular orientation six weeks after implantation. (13) Immunocytochemical analysis using troponin I antibodies (brown) identifies cardiac fibers within the implanted constructs six weeks after implantation. (C) Cardiac cell implant in control group shows fibrosis and necrotic debris (d) at six weeks. (D) Cloned skeletal muscle cell implants show well-organized bundle formation (12 weeks). (E) Retrieved skeletal cell implant with polymer fibers (arrows) at 12 weeks. (F) Immunohistochemical analysis using sarcomeric tropomyosin antibodies (brown) identifies skeletal fibers within the implanted second-set constructs 12 weeks after implantation. (G) Retrieved cloned skeletal cell implants show spatially oriented muscle fiber 12 weeks after implantation. (H, I) Retrieved control skeletal cell implants show fibrosis with increased inflammatory reaction (arrows) and necrotic debris at 12 weeks (J) Immunocytochemical analysis using CD4 antibodies (brown) identifies CD4+T cells within the implanted control cardiac construct six weeks after implantation. Bars, 100 μm (A, B, E); 200 μm (C, G, I, J); 800 μm (D, F, H). Panels (A, C-E, G-I), H&E staining.

FIG. 5—RT-PCR and western blot analyses. Semi-quantitative RT-PCR products indicate specific mRNA in the retrieved skeletal muscle tissue (A) and cardiac muscle tissue (B). Western blot analysis of the implants confirmed the expression of specific proteins in the skeletal muscle tissues (C) and cardiac muscle tissues (D). CL6 and CL12, cloned group at 6 and 12 weeks, respectively, CO6 and CO12, control group at 6 and 12 weeks, respectively.

FIG. 6—Tissue-engineered renal units. (A) Illustration of renal unit and units retrieved three months after implantation. (B) Unseeded control. (C) Seeded with allogeneic control cells. (D) Seeded with cloned cells, showing the accumulation of urinelike fluid.

FIG. 7—Characterization of renal explants. (A, B) Cloned cells stained positively with synaptopodin antibody (green; A) and AQP1 antibody (green; B). (C) The allogeneic controls displayed a foreign-body reaction with necrosis. (D) Cloned explant shows organized glomeruli-like structures. Vascular tufts (v); visceral epithelium (arrow). H&E. (E) Organized tubules (arrows) were shown in the retrieved cloned explant. (F) Immunohistochemical analysis using Factor VIII antibodies (brown) identifies vascular structures. (G) There was a clear unidirectional continuity between the mature glomeruli, their tubules, and the polycarbonate membrane. Bars, 100 μm (B, D-F); 200 μm (A); 800 μm (C).

FIG. 8—RT-PCR analyses (top panel) confirming the transcription of AQP1, AQP2, Tamm-Horsfall, and synaptopodin genes exclusively in the cloned group (CIs). Western blot analysis (bottom panel) confirms high protein levels of AQP1 and AQP2 in the cloned group, whereas expression intensities of CD4 and CDS were significantly higher in the unseeded and allogeneic control groups (Co 1 and Co 2, respectively). Each lane represents a different cloned tissue.

FIG. 9—Elispot analyses of the frequencies of T cells that secrete IFNy after primary and secondary stimulation with allogeneic renal cells, cloned renal cells, or nuclear donor fibroblasts. The presented wells are single representatives of the duplicate wells for each responder-stimulator combination.

### DESCRIPTION OF THE INVENTION

The present invention produces novel and improved methods for producing cells and tissues suitable for therapeutic transplant to humans and non-human mammals in need of such transplant therapy. The present invention provides methods whereby cells and tissues suitable for therapeutic transplant to humans and non-human mammals are obtained from embryos produced by nuclear transfer cloning, or from embryonic stem cells or other stem cells obtained from such embryos. The present invention also provides methods whereby cells and tissues suitable for therapeutic transplant to humans and non-human mammals are obtained from embryos produced by androgenesis or gynogenesis. The present invention also provides methods for producing model systems for studying the biochemical, metabolic, and physiological interactions that control embryogenesis, and the role played by genetic and epigenetic factors in determining the course of embryogenesis.

Cells and Tissues from Embryos Produced by Nuclear Transfer Cloning.

In one embodiment of the present invention, cells having significant therapeutic potential for use in cell therapy are derived from early stage embryos that are produced by nuclear transfer cloning. This is a cloning method that comprises transferring a donor cell, or the nucleus or chromosomes of such a cell, into an oocyte, and coordinately removing the oocyte genomic DNA, to produce an embryo from which cells or tissues suitable for transplant can be derived, as described, for example, in co-owned and co-pending U.S. application Ser. No. 09/655,815 filed Sep. 6, 2000, and Ser. No. 09/797,684 filed Mar. 5, 2001, the disclosures of which are incorporated herein by reference in their entirety.

To provide histocompatible cells and tissues suitable for transplant, nuclear transfer cloning is carried out using a germ or somatic donor cell from the human or non-human mammal that is the transplant recipient, as described in the aforementioned co-pending U.S. applications. Alternatively, cells and tissues suitable for transplant may be obtained by performing nuclear transfer cloning with a donor cell having DNA comprising MHC alleles that match those of the transplant recipient. Cells and tissues derived from an embryo produced by such a method are not syngenic with, but have the same MHC antigens as the cells of the transplant recipient, so that rejection by the recipient is muted, as described in the co-pending application, “A Bank of Nuclear Transfer-Generated Stem Cells for Transplantation Having Homozygous MHC Alleles, and Methods for Making and Using Such a Stem Cell Bank, filed May 24, 2002, the disclosure of which is incorporated herein by reference in its entirety.

According to the present invention, nuclear transfer embryos are produced by known methods, e.g. those disclosed in any of U.S. Pat. Nos. 6,252,133; 6,235,970; 6,235,969; 6,215,041; 6,147,276; 5,994,619 and 5,945,577, all of which are incorporated by reference in their entireties herein. In performing nuclear transfer cloning to produce cells and tissues for transplant, both the nuclear donor cell and the oocyte or other recipient cell may be from any species of mammal. For example, the donor and recipient cells may be from any species of rodent, ungulate, lagomorph, or primate. Examples of rodent species from which donor and recipient cells may be obtained are mouse, rat, guinea pig, hamster and gerbil. Examples of ungulate that may be used as sources donor and recipient cells include bovines, ovines, caprines, equines, and bison (buffalo). Rabbits are an example of a lagomorph species may be used as source of donor cells. Examples of primate species from which donor and recipient cells may be obtained are humans, chimpanzees, baboons, cynomolgus monkeys, and any other New or Old World monkeys.

As described in co-owned and co-pending U.S. application Ser. No. 09/685,061 filed Oct. 6, 2000, Ser. No. 09/809,018 filed Mar. 16, 2001, and Ser. No. 09/874,040 filed Jun. 6, 2001, the disclosures of which are incorporated herein by reference in their entirety, the nuclear donor cell and the oocyte or other recipient cell used for nuclear transfer may be of the same species, or they may be of different species. For example, the nuclear donor cell and the recipient oocyte may both be from the same bovine species, or from humans. Alternatively, the nuclear donor cell may be from a sheep or a human, and the recipient cell, e.g., oocyte, may be from a cow or a rabbit.

As described in the above-identified patents and co-pending applications, nuclear transfer cloning is effected by introducing a donor cell, or the nucleus or chromosomes of a donor cell, into a recipient cell that is typically an oocyte, blastomere or other embryonic cell. As the nuclear transfer recipient cell is frequently an oocyte, the present application sometimes refers to the nuclear transfer recipient cell as an oocyte; however, the present invention includes providing and using cells and tissue for transplant that are obtained by nuclear transfer methods wherein the transfer recipient is a blastomere or other embryonic cell. Great efforts are presently being made to develop methods for inducing a cell to undergo “reprogramming,” a de-differentiating process whereby a cell committed to a given lineage of differentiation acquires the ability to divide and give rise to cells that differentiate to one or more different lineages. Such methods may comprise transferring cytoplasm, a fraction of the cytoplasm, or one or more factors present in the cytoplasm, of an oocyte, blastomere or other embryonic cell into a differentiated somatic cell to effect its reprogramming, as described in co-owned and co-pending U.S. application Ser. No. 09/736,268, filed Dec. 15, 2000, the disclosure of which is incorporated herein by reference in its entirety. Accordingly, the present invention also includes providing and using cells and tissue for transplant that are obtained by a reverse nuclear transfer method whereby a committed donor cell is induced to de-differentiate into a pluripotent or totipotent cell cpable of dividing and giving rise to cells that differentiate to a lineage different from that to which the nuclear donor cell was originally committed.

As described in the above-identified patents and co-pending applications, the somatic donor cell used for nuclear transfer to produce a nuclear transplant embryo according to the present invention can be of any germ cell or somatic cell type in the body. For example, the donor cell can be a germ cell or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, stomach, intestines, lung, pancreas, cornea, skin, gallbladder, ovary, testes, kidneys, heart, bladder, and urethra.

As used herein, enucleation refers removal of the genomic DNA from an cell, e.g., from a recipient oocyte. Enucleation therefore includes removal of genomic DNA that is not surrounded by a nuclear membrane, e.g., removal of chromosomes at a metaphase plate. As described in the above-identified patents and co-pending applications, the recipient cell can be enucleated by any of the known means either before, concomitant with, or after nuclear transfer. For example, a recipient oocyte may be enucleated when the oocyte is arrested at metaphase II, when oocyte meiosis has progressed to telophase, or when meiosis has completed and the maternal pronucleus has formed.

As described in the above-identified patents and co-pending applications, the donor genome may be introduced into the recipient cell by injection or fusion of the nuclear donor cell and the recipient cell, e.g., by electrofusion or by Sendai virus-mediated fusion. Suitable testing and microinjection methods are well known and are the subject of numerous issued patents. The donor cell, nucleus, or chromosomes can be from a proliferative cell (e.g., in the G1, G2, S or M cell cycle stage); alternatively, they may be derived from a quiescent cell (in G0).

As described in the above-identified patents and co-pending applications, the recipient cell may be activated prior to, simultaneous with, and/or after nuclear transfer.

Direct Harvest of Therapeutic Cells and Tissue from an Embryo

Cells or tissue for transplant can be obtained from a nuclear transfer embryo that has been cultured in vitro to form a gastrulating embryo of from about one cell to about 6 weeks of development. For example, cells or tissue for transplant may be obtained from an embryo of from 15 days to about four-weeks old. Alternatively, in the case of non-human NT embryos, cells or tissue for transplant may be obtained from a gastrulating embryo of up to six weeks old, or older, by transferring an NT embryo into a suitable maternal recipient and allowing it to develop in utero for up to six weeks, or longer. Thereupon, it may be harvested from the uterus of the maternal recipient and used as a source of cells or tissues for transplant.

The therapeutic cells that are obtained from a gastrulating embryo at a developmental stage of from one cell to up to six weeks of age can be pluripotent stem cells and/or cells that have commenced becoming committed to a particular cell lineage, e.g., hepotocytes, myocardiocytes, pancreatic cells, hemagioblasts, hematopoietic progenitors, CNS progenitors and others.

Generation of Therapeutic Cells and Tissue from Pluripotent Embryonic Stem Cells

In addition to obtaining cells and tissue for transfer from a gastrulating embryo as described above, cells and tissues for therapeutic transfer according to the invention can be generated from pluripotent and/or totipotent stem cells derived from a nuclear transfer embryo produced by the methods of the invention. As described in co-pending U.S. application Ser. Nos. 09/655,815 and 09/797,684, the disclosures of which are incorporated herein by reference, pluripotent and totipotent stem cells produced by nuclear transfer methods according to the present invention can be cultured using methods and conditions known in the art to generate cell lineages that differentiate into specific, recognized cell types, including germ cells. These methods comprise:
a) inserting a donor cell, or the nucleus or chromosomes of such a cell, into an oocyte or other suitable recipient cell, and coordinately removing the genomic DNA of the oocyte or other recipient cell to produce a nuclear transfer embryo; and
b) generating stem cells and/or differentiated cells or tissue needed for transplant from said embryo having the genomic DNA of the donor cell.
   Such a method can be used to generate generate pluripotent stem cells and/or totipotent embryonic stem (ES) cells. Pluripotent stem cells produced in this manner can be cultured to generate cell lineages that differentiate into specific, recognized cell types. The totipotent ES cells produced by nuclear transfer have the capacity to differentiate into every cell type of the body, including the germ cells. For example, the pluripotent and/or totipotent stem cells derived from a nuclear transfer embryo can differentiate into cells selected from the group consisting of immune cells, neurons, skeletal myoblasts, smooth muscle cells, cardiac muscle cells, skin cells, pancreatic islet cells, hematopoietic cells, kidney cells, and hepatocytes suitable for transplant according to the present invention. Because the pluripotent and totipotent stem cells produced by such methods have the patient's own genomic DNA, the differentiated cells and tissues generated from these stem cells are nearly completely autologous—all of the cells' proteins except those encoded by the cells' mitochondria, which derive from the oocyte, are encoded by the patient's own DNA. Accordingly, differentiated cells and tissues generated from the stem cells produced by such nuclear transfer methods can be used for transplantation without triggering the severe rejection response that results when foreign cells or tissue are transplanted.

In preparing the pluripotent and totipotent stem cells having primate genomic DNA according to the present invention, one can employ the methods described in James A. Thomson's U.S. Pat. No. 6,200,806, “Primate Embryonic Cells,” issued Mar. 13, 2001. For example, the Thomson patent describes a method for preparing human pluripotent stem cells comprising:
a) isolating a human blastocyst;
b) isolating cells from the inner cell mass of the blastocyst;
c) plating the inner cell mass cells on embryonic fibroblasts so that inner-cell mass-derived cell masses are formed;
d) dissociating the mass into dissociated cells;
e) replating the dissociated cells on embryonic feeder cells;
f) selecting colonies with compact morphologies and cells with high nucleus to cytoplasm ratios and prominent nucleoli; and
g) culturing the selected cells to generate a pluripotent human embryonic stem cell line.
   The disclosure of Thomson's U.S. Pat. No. 6,200,806 is incorporated herein by reference in its entirety.

A method for inducing the differentiation of pluripotent human embryonic stem cells into hematopoietic cells useful for transplant according to the present invention is described in U.S. Pat. No. 6,280,718, “Hematopoietic Differentiation of Human Pluripotent Embryonic Stem Cells,” issued to Kaufman et al. on Aug. 28, 2001, the disclosure of which is incorporated herein by reference in its entirety. The method disclosed in the patent of Kaufman et al. comprises exposing a culture of pluripotent human embryonic stem cells to mammalian hematopoictic stromal cells to induce differentiation of at least some of the stem cells to form hematopoietic cells that form hematopoietic cell colony forming units when placed in methylcellulose culture.

Generation of “Hyper-Young” Cells and Tissue for Transplant

Nuclear transfer cloning methods can also be employed to generate “hyper-young” embryos from which cells or tissues suitable for transplant can be derived. Methods for generating rejuvenated, “hyper-youthful” stem cells and differentiated somatic cells having the genomic DNA of a somatic donor cell of a human or non-human mammal are described in co-owned and co-pending U.S. application Ser. No. 09/527,026 filed Mar. 16, 2000, Ser. No. 09/520,879 filed Apr. 5, 2000, and Ser. No. 09/656,173 filed Sep. 6, 2000, the disclosures of which have been incorporated herein by reference in their entirety. For example, rejuvenated, “hyper-youthful” cells having the genomic DNA of a human or non-human mammalian somatic cell donor can be produced by a method comprising:
a) isolating normal, somatic cells from a human or non-human mammalian donor, and passaging or otherwise inducing the cells into a state of checkpoint-arrest, senescence, or near-senescence,
b) transferring such a donor cell, the nucleus of said cell, or chromosomes of said cell, into a recipient oocyte, and coordinately removing the oocyte genomic DNA from the oocyte, to generate an embryo; and
c) obtaining rejuvenated cells from said embryo having the genomic DNA of the donor cell.

The rejuvenated cells obtained from the embryo can be pluripotent stem cells or partially or terminally differentiated somatic cells. As described in the above-identified co-pending applications, rejuvenated pluripotent and/or totipotent stem cells can be generated from a nuclear transfer embryo by a method comprising obtaining a blastocyst, an embryonic disc cell, inner cell mass cell, or a teratoma cell using said embryo, and generating the pluripotent and/or totipotent stem cells from said blastocyst, inner cell mass cell, embryonic disc cell, or teratoma cell.

As described in the above-identified co-pending applications, rejuvenated cells derived from a nuclear transfer embryo according to the present invention are distinguished in having telomeres and proliferative life-spans that that are as long as or longer than those of age-matched control cells of the same type and species that are not generated by nuclear transfer techniques. In addition, the nucleotide sequences of the tandem (TTAGGG)ₙ repeats that comprise the telomeres of such rejuvenated cells are more uniform and regular; i.e., have significantly fewer non-telomeric nucleotide sequences, than are present in the telomeres of age-matched control cells of the same type and species that are not generated by nuclear transfer. Such rejuvenated cells are also have patterns of gene expression that are characteristic of youthful cells; for example, activities of EPC-1 and telomerase in such rejuvenated cells are typically greater than EPC-1 and telomerase activities in age-matched control cells of the same type and species that are not generated by nuclear transfer techniques. Moreover, the immune systems of cloned animals produced by nuclear transfer procedures are shown to be enhanced, i.e., to have greater immune responsiveness, than those of animals that are not generated by nuclear transfer techniques. When introduced into a subject, e.g., a human or non-human mammal in need of cell therapy, the cells and tissues derived from such “hyper-young” embryos are capable of efficiently infiltrating and proliferating at a desired target site, e.g., heart, brain, liver, bone marrow, kidney or other organ that requires cell therapy. Hematopoietic progenitor cells derived from such “hyper-young” embryos are expected to infiltrate into a subject and rejuvenate the immune system of the individual by migrating to the immune system, ie., blood and bone marrow. Similarly, CNS progenitor cells derived from such “hyper-young” embryos are expected to preferentially migrate to the brain, e.g., that of a Parkinson's, Alzheimer's, ALS, or a patient suffering from age-related senility.

Histocompatible Cells for Transfer Produced by Androgenesis and Gynogenesis.

Methods for producing haploid and diploid gynogenetic embryos suitable as sources of syngenic cells and tissues for transplant are known; for example, such methods are described in co-owned U.S. Provisional Application No. 60/163,086, filed Nov. 2, 1999, and in co-owned and co-pending U.S. Non-Provisional application Ser. No. 09/995,659, both disclosures of which are incorporated herein by reference in their entirety.

Methods For Producing Androgenetic Embryos

Histocompatible cells and tissues for transplant can be obtained by constructing haploid and diploid androgenetic embryos, using donor gametes from the male that is to receive the transplant. The embryos produced by this method have the genomic DNA of the transplant recipient, and cells and tissues for transplant derived from the embryo are relatively histocompatible with the recipient.

I. Producing Haploid, Androgenetic Embryos:

(a) In one embodiment of the invention, the maternal genomic DNA is removed from an unfertilized oocyte, and the oocyte is fertilized by a single sperm cell or nucleus to produce an oocyte having a haploid, all-male genome. The fertilized oocyte is then allowed to divide mitotically to produce a haploid androgenetic embryo. The oocyte can be fertilized before or after removal of the maternal genomic DNA.

(b) In another embodiment, the germinal vesicle (G2 immature oocyte nucleus) is removed from an immature oocyte by micromanipulation, and a spermatogonium in G2 or a primary spermatocyte is introduced into the enucleated oocyte. The reconstructed oocyte is then maintained under conditions that support oocyte maturation, with the result that the paternal DNA undergoes meiosis I and arrests at metaphase II with formation of a metaphase plate that contains exclusively paternal chromosomes. Activation of the oocyte leads to generation of a haploid, all-male embryo in an androgenetic process analogous to parthenogenesis.

(c) Alternatively, a metaphase II oocyte containing exclusively paternal chromosomes can be constructed as described above and inseminated with a second sperm cell or nucleus by IVF or ICSI, whereupon removal of one of the male pronuclei results in production of a haploid, all-male embryo.

II. Producing Diploid, Androgenetic Embryos with Identical Homologous Chromosomes

The present invention also provides means for producing diploid, androgenetic, uniparental embryos comprised of cells in which the two homologous sets of chromosomes are identical to each other. This form of the invention comprises introducing a single haploid sperm cell or nucleus into an oocyte, removing the maternal genomic DNA from the oocyte, allowing the sperm DNA to be replicated, and manipulating the embryo to obtain a single-cell embryo (i.e., a zygote) containing two identical copies of each paternal chromosome.

(a) For example, in one embodiment, the invention comprises fertilizing an oocyte with a single sperm cell or nucleus, removing the maternal genomic DNA from the oocyte, allowing the oocyte to undergo mitosis and cleavage to generate a two-cell embryo, each cell of which has the haploid, all-male genome of the fertilizing sperm cell, and fusing the cells of the 2-cell embryo to produce a diploid, androgenetic, uniparental zygote.

(b) Alternatively, the maternal genomic DNA can be removed from the oocyte before fertilizing the oocyte with a single sperm cell or nucleus to produce an oocyte having a haploid, all-male genome. As before, the fertilized oocyte is then allowed to divide mitotically to generate a 2-cell embryo, each cell having a haploid, all-male genome, and the cells of the 2-cell embryo are fused to produce a diploid, androgenetic, uniparental zygote.

(c) In another embodiment, an oocyte is fertilized or a sperm cell or nucleus is microinjected into oocyte, the maternal chromosomes are removed, and the chromosomes contributed by the sperm are diploidized by blocking karyokinesis and cytokinesis of the first mitotic division to produce a diploid, androgenetic, uniparental zygote. Diploidization can be effected by commonly used methodology; e.g., by heat-shock, or by incubating the oocyte for a defined period in medium comprising a microfilament inhibitor such as cytochalasin B or a microtubule inhibitor such as colchicine.

III. Diploid Androgenetic Embryos and Embryonic Stem Cells with Non-Identical Homologous Chromosomes

The present invention also provides means for producing diploid androgenetic, uniparental or bi-parental embryos made up of cells in which the two chromosomes of each homologous chromosome pair are not identical to each other. This method comprises introducing two complete, non-identical, haploid sets of chromosomes of male-origin into an oocyte and removing the maternal genomic DNA from the oocyte to produce a zygote having all-male genomic DNA packaged in two non-identical sets of homologous chromosomes.

(a) One embodiment of the invention comprises introducing a single, diploid male germ cell or nucleus into an oocyte and removing the maternal genomic DNA from the oocyte to produce a uniparental diploid cell having all-male genomic DNA in two non-identical sets of chromosome. For example, the method can be performed by injecting a diploid male germ cell (e.g., a secondary spermatocyte) into a mammalian oocyte before or after removal of the oocyte's maternal DNA. Manipulation following injection of the diploid male germ cell can be carried out in the presence of a microfilament inhibitor, e.g., cytochalasin B, to prevent the paternal chromosomes from being extruded from the oocyte as a “paternal” polar body during activation.

(b) In another embodiment, a spermatogonium in G2 or a primary spermatocyte is introduced into an immature oocyte before or after removal of the germinal vesicle (G2 immature oocyte nucleus) from the oocyte by micromanipulation. The reconstructed oocyte is then maintained under conditions that support oocyte maturation, with the result that the paternal DNA undergoes meiosis I and arrests at metaphase II following formation of a metaphase plate that contains exclusively paternal chromosomes. The oocyte is then fertilized with another sperm cell or nucleus by in vitro fertilization (IVF) or intracytoplasmic sperm injection (ICSI) to generate a diploid zygote containing only male-derived genomic DNA.

(c) The method of the invention can also be performed by injecting two post-meiotic, haploid male gametes into the cytoplasm of a mature, metaphase II mammalian oocyte before or after removal of the maternal chromosomal DNA. For example, the maternal chromosomal DNA is removed prior to injecting the two haploid male gametes, or immediately after injecting the two haploid male gametes, while the oocyte is still in metaphase arrest. Alternatively, two haploid male gametes are injected into a metaphase II oocyte and the maternal genomic DNA is removed shortly after activation, during the anaphase and/or telophase of maternal chromosome separation. In another embodiment, two haploid male gametes are injected into a metaphase II oocyte, and the reconstructed zygote is allowed to progress to the first zygotic interphase, at which time the pronucleus containing the maternal genomic DNA is removed. At this stage, the genomic DNA within the oocyte is present in 3 pronuclei—2 of paternal and one of maternal origin. The pronuclei in the oocyte can be visualized by methods known to those in the art; for example, by phase contrast microscopy, or by differential interference contrast microscopy (DIC). In primates, the two paternal pronuclei can be distinguished from the maternal pronucleus by their association with the sperm mid-piece and the remainder of the sperm tail. The maternal pronucleus is removed from the zygote by micromanipulation in the presence of cytochalasin B, using established techniques. In producing all-male embryos of species for which the maternal and paternal pronuclei are not easily distinguished, multiple embryos can be prepared and a pronucleus can be removed from each, with a 67% likelihood of producing a diploid, androgenetic zygote.

(d) The present invention can also be performed by pronuclear exchange. In this embodiment, oocytes are inseminated by IVF or ICSI to produce zygotes containg male and female pronuclei. A female pronucleus is then removed from a recipient zygote by micromanipulation and is replaced by a male pronucleus isolated from another (donor) zygote, to produce a reconstructed zygote containing two male pronuclei.

(e) In another embodiment of the invention, mature, metaphase II mammalian oocytes are enucleated and are inseminated in vitro under conditions that favor dispermic fertilization, to produce oocytes containing genomic DNA contributed by two haploid sperm. Conditions that influence the number of sperm by which oocytes are fertilized in vitro may be manipulated to increase the frequency of dispermy. Such conditions include sperm concentration, the concentration of capacitation inducer, e.g., caffeine, heparin, heparan sulfate or other glycosaminoglycans, the duration of the such insemination conditions, and the concentration of sperm motility enhancers and antioxidants, e.g., epinephrine, hypotaurin and penicillamin. The maternal genomic DNA is removed from the oocytes before or after fertilization. For example, condensed maternal chromosomes at the metaphase II plate are removed by micromanipulation in the presence of cytochalasin B prior to or shortly after fertilization. Alternatively, the fertilized oocyte is allowed to complete meiosis, and the pronucleus containing the maternal genomic DNA is then removed from the zygote by micromanipulation in the presence of cytochalasin B.

In producing androgenetic and gynogeneti embryos, the oocyte can be of the same species as the cell that contributes the chromosomes, or it can be of a different mammalian species, as in nuclear transfer,

In the embodiments described above, the oocyte can be fertilized by letting the sperm contact the oocyte surface, or by injecting the sperm or sperm nucleus into the oocyte. Introduction of the sperm into the oocyte by contact fertilization can be performed when the oocyte that is a mature, metaphase II oocyte. When the sperm is microinjected into the oocyte, the oocyte can be an immature, pre-metaphase II oocyte, it can be a mature, metaphase II oocyte, or it can be post-metaphase II; however, the oocyte that is used should be at stage is competent to induce the male-derived chromosomes to undergo mitosis. The sperm chromosomes can be introduced by injecting a complete sperm cell into the oocyte; alternatively, good results are also obtained by injecting an incomplete sperm cell, e.g., the headpiece, provided that the portion that is injected comprises a complete, 1N set of chromosomes. The oocyte can be enucleated before or after fertilization or injection of the sperm. For example, the maternal chromosomes can be removed when the oocyte is arrested at metaphase II, when oocyte meiosis has progressed to telophase, or when meiosis has completed and the maternal pronucleus has formed.

Fusion of the cells of a 2-cell stage embryo to form a zygote can be effected using any of the known techniques for inducing cell-fusion; for example, by incubating the cells with Sendai virus, or by subjecting the cells to an electromagnetic pulse.

In the techniques that combine haploid genomes of two different male cells, the two haploid male genomes can be from the same male individual, or from different male individuals. In producing cells and tissues useful for therapy, e.g., for transplantation to a male individual in need of such therapy, can obtain both cells from the same male in order to produce cells and tissues that are immune-compatible with the individual in need of treatment; or the haploid gametes can be from two different males; e.g., in a study of how the different structures and genetic sequences of the chromosomes of the two different males interact with each other and with factors in the cytosol of the embryonic cells to affect embryonic development.

Haploid male gametes that are introduced into the oocyte are selected from the group consisting of mature spermatozoa, elongated spermatids and round spermatids. Mature, metaphase II oocytes of human and non-human primates are generally activated by injection of any of these male gamete cells. Using oocytes of other species, such as cattle, the injected oocytes may have to be artificially activated in order to start embryonic development.

Spontaneous diploidization may occur when cells of haploid androgenetic blastocysts are explanting into tissue culture and cultured, leading to generation of pluripotent, homozygous, diploid cell lines (see Kaufman et al., J. Embryol. Exp. Morphol. (1983)). Diploidization can be in androgenesis by induced by removing the maternal pronucleus and replacing it with a haploid pronucleus; allowing the male DNA to replicate, and incubating egg with cytochalasin B to prevent karyokinesis (separation of chromosomes) and cytokinesis (division of cytoplasm) of 1^{st} mitotic division. Alternatively, diploidization can be induced removing the maternal pronucleus and replacing with a haploid pronucleus; allowing the male DNA to replicate, and subjecting the egg to heat shock or to a 240V DC-pulse to prevent karyokinesis (separation of chromosomes) and cytokinesis (division of cytoplasm) of 1^{st} mitotic division. (See Landa et al., Folia Biol. (Praha) (1990) 36(3-4):145-152).

In alternative embodiments of the methods in which two haploid male gametes are injected into an oocyte to produce a diploid zygote, haploid, androgenetic zygotes can be produced by injecting a single post-meiotic, haploid male gamete into the cytoplasm of the mature, metaphase II mammalian oocyte, and by removing the maternal DNA from the oocyte as described above.

After constructing a replicating, diploid embryo, the embryo is cultured in vitro or in vivo by known methods to obtain ES cells. For example, diploid, androgenetic embryonic stem cells are generated from a diploid, androgenetic zygote produced by the above-described methods, by permitting the androgenetic zygote to develop into a blastocyst having an inner cell mass, isolating cells of the inner cell mass, and culturing them under conditions suitable for producing embryonic stem cells.

The imprinting of male chromosomes may reduce the ability of an androgenetic embryos to develop to a stage at which a desired cell or tissue can be obtained. In this case, it is possible to “rescue” cells of an androgenetic embryo, i.e., to promote their further development, by the following methods:
(a) introduce the inner cell mass of an androgenetic embryo into a normal blastocyst (see Barton et al., Development (1991), 113(2):679-687).
(b) produce an aggregation chimera by combining the androgenetic embryo with one or more normal embryos (see Mann et al., Development (1991), 113(4): 1325-1333).
(c) generate androgenetic ES cells, and inject these into a normal blastocyst to generate a chimera (see Mann et al., Development (1991), 113(4):1325-1333).
(d) produce an aggregation chimera by combining an androgenetic embryo with one or more tetraploid embryos at the 4- to 8-cell stage (see Goto et al., Development (1999), 125:3353-3363)
(e) use androgenetic ES cells to generate embryoids from which the cells or tissues for transplant are derived (see Szabo et al., Development (1994), 120:1651-1660).
(f) use a cell of an androgenetic embryo, e.g., a blastomere, ICM cell, or trophoblast, as a donor cell for nuclear transfer to produce an embryo from which the cells or tissues for transplant are derived (see Hoppe et al., PNAS (1982) 79(6):1912-1916).
(g) use an androgenetic ES cell as a donor cell for nuclear transfer to produce an embryo from which the cells or tissues for transplant are derived.
(h) use an androgenetic somatic cell of and androgenetic/wild-type or androgenetic/tetraploid chimeric embryo as the donor cell for nuclear transfer to produce an embryo from which the cells or tissues for transplant are derived.
   Genetically Modified Cells and Tissues for Transplant

Cells and tissues produced for transplant according to the present invention can be genetically altered by any known means. Genetically modified cells and tissues for transplant can be obtained by performing nuclear transfer with a genetically modified nuclear donor cell to produce nuclear transfer embryo made up of genetically modified cells. Alternatively, cells and tissues for transplant can be genetically modified after they are derived from a nuclear transfer embryo.

In some cases, it may be desirable for the cells to express or not express a desired DNA sequence. This may be accomplished by genetically modifying the genome of the donor cell used to produce the nuclear transfer embryo. In some instances, particularly in the case of multiple gene modifications or gene knockout this may be accomplished by repeated nuclear transfer procedures wherein the genome of a donor cell is modified. used to produce an NT embryo and cells derived from this NT embryo or fetus resulting therefrom subjected to a second genetic modification and the resultant cells used as donor cells to produce other nuclear transfer embryos containing both genetic modifications. This process may be repeated indefinitely until NT embryos containing cells having all the desired genetic modifications are obtained.

Genetic Modification to Produce a Lineage-Deficient Donor Cell

As described in co-owned and co-pending U.S. application Ser. No. 09/685,061 filed on Oct. 6, 2000, the disclosure of which is incorporated herein in its entirety, a nuclear transfer donor cell e.g., a human cell, can be genetically modified such that it is lineage deficient, so that when it is used for nuclear transfer it is unable to give rise to a viable offspring. This is desirable especially in the context of human nuclear transfer embryos, wherein for ethical reasons, production of a viable embryo may be an unwanted outcome. This can be effected by genetically engineering a human cell such that it is incapable of differentiating into specific cell lineages when used for nuclear transfer. In particular, cells may be genetically modified such that when used as nuclear transfer donors the resultant “embryos” do not contain or substantially lack at least one of mesoderm, endoderm or ectoderm tissue It is anticipated that this can be accomplished by knocking out or impairing the expression of one or more mesoderm, endoderm or ectoderm specific genes. Examples thereof include:
Mesoderm: SRF, MESP-1, HNF-4, beta-I integrin, MSD;
Endoderm: GATA-6, GATA-4;
Ectoderm: RNA helicase A, H beta 58.

The above list is intended to be exemplary and non-exhaustive of known genes which are involved in the development of mesoderm, endoderm and ectoderm. The generation of mesoderm deficient, endoderm deficient and ectoderm deficient cells and embryos has been previously reported in the literature. See, e.g., Arsenian et al, EMBO J., Vol. 17(2):6289-6299 (1998); Saga Y, Mech. Dev., Vol. 75(1-2):53-66 (1998); Holdener et al, Development, Voll. 120(5):1355-1346 (1994); Chen et al, Genes Dev. Vol. 8(20):2466-2477 (1994); Rohwedel et al, Dev. Biol., 201(2):167-189 (1998) (mesoderm); Morrisey et al, Genes, Dev., Vol. 12(22):3579-3590 (1998); Soudais et al, Development, Vol. 121(11):3877-3888 (1995) (endoderm); and Lee et al, Proc. Natl. Acad. Sci. USA, Vol. 95:(23):13709-13713 (1998); and Radice et al, Development, Vol. 111(3):801-811 (1991) (ectoderm).

In general, a desired somatic cell, e.g., a human keratinocyte, epithelial cell or fibroblast, will be genetically engineered such that one or more genes specific to particular cell lineages are “knocked out” and/or the expression of such genes significantly impaired. This may be effected by known methods, e.g., homologous recombination. A preferred genetic system for effecting “knock-out” of desired genes is disclosed by Capecchi et al, U.S. Pat. Nos. 5,631,153 and 5,464,764, which reports positive-negative selection (PNS) vectors that enable targeted modification of DNA sequences in a desired mammalian genome. Such genetic modification will result in a cell that is incapable of differentiating into a particular cell lineage when used as a nuclear transfer donor.

This genetically modified cell will be used to produce a lineage-defective nuclear transfer embryo, i.e., that does not develop at least one of a functional mesoderm, endoderm or ectoderm. Thereby, the resultant embryos, even if implanted, e.g., into a human uterus, would not give rise to a viable offspring. However, the ES cells that result from such nuclear transfer will still be useful in that they will produce cells of the one or two remaining non-impaired lineage. For example, an ectoderm deficient human nuclear transfer embryo will still give rise to mesoderm and endoderm derived differentiated cells. An ectoderm deficient cell can be produced by deletion and/or impairment of one or both of RNA helicase A or H beta 58 genes.

Cell Therapy

Cells and tissues produced according to the invention are useful in treating any disorder that is treatable by cell therapy. Because of their very early differentiation status and their youthful, embryonic-like state, the cells and tissues of the present application will efficiently migrate and infiltrate target sites, such as areas of tissue injury. Particularly, these cells are able when introduced into a subject, e.g., a human or animal, to infiltrate and proliferate at a desired target site, e.g., heart, brain, liver, bone marrow, kidney or other organ that requires cell therapy. For example, it is anticipated that such hematopoietic progenitors will infiltrate into a subject and will rejuvenate the immune system of the individual by migrating to the immune system, i.e., blood and bone marrow. Alternatively, in the case of CNS progenitor such cells should preferentially migrate to the brain, e.g., that of a Parkinson's, Alzheimer's, ALS, or a patient suffering from age-related senility.

Cells of a particular lineage may be selected by known methods. Cells which have commenced becoming committed to desired cell lineages contained in embryos may be identified, e.g., by assaying for the expression of cell markers characteristic of a particular cell lineage, e.g., hepatocyte markers in situations wherein cell therapy for treating the liver is warranted or pancreatic markers where the subject has a disorder involving the pancreas, e.g., type I or type II diabetes.

Therapeutic applications wherein cells produced according to the invention are useful for cell therapy includes transplantation, cancer, autoimmune diseases of all kinds, proliferative disorders, inflammatory disorders, neurological disorders, age-related disorders, allergic. disorders, immune disorders, viral infections, burn, trauma, other conditions involving tissue injury, and other conditions wherein replacement cells are desirable.

Specific examples include lupus, diabetes, myasthenia gravis, rheumatoid arthritis, ALS, Parkinson's disease, Alzheimer's disease, Huntington's disease, paralysis, multiple sclerosis, thyroiditis, AIDS, psoriasis, psoriatic arthritis, pancreatitis, hematologic malignancies, non-specific cell damage associated with radiotherapy or chemotherapy, cardiac injuries, e.g., associated with heart attack, Sjogren's syndrome, and many others.

Cell therapy will be effected by known methods. Typically the cells will be administered paremerally, e.g., via intravenous injection. The cells will preferably be in solution, e.g. buffered saline. The number of cells administered will be an amount effective to treat the particular condition. It may be beneficial also for the cells to express a marker, e.g., green fluorescent protein (GFP), while allowing for the detection of sites) and number of cells which have become stably engrafted in the subject. The use. of GFP and variants thereof to detect specific. cells is well known in the art.

In some in instances, it may be necessary to repeatedly administer the cells, e.g., in the case of chronic diseases such as autoimmune disorders or cancer. It may also be necessary in instances where the initial cells do not become stably engrafted at the desired target site.

Model Systems for Developing and Testing Cell Transplant Therapies.

The present invention further relates to methods for producing and using model embryonic, fetal, and developed animal systems having defined genetic makeup that are of use in developing and testing methods for cell and tissue therapy, and as model systems for studying imprinting, reprogramming, rejuvenation, and other biochemical, metabolic, and physiological phenomena associated with embryogenesis and development.

The embryos, pluripotent and totipotent stem cells, and the differentiated cells and tissues that are obtained or generated from these for therapeutic transplant according to the present invention, are produced and isolated under Good Manufacturing Practices (GMP) conditions.

Although not limiting, the scope and spirit of the invention are illustrated by reference to the following discussion and examples.

### EXAMPLE 1

This is a prophetic example that demonstrates the therapeutic utility of the present invention. In practice, the exemplified method is an acceptable way to provide cells and tissues for transplant to a non-human-mammal, but would not be undertaken to treat a human patient because it requires destruction of a viable embryo.

A human NT embryo is produced by introducing a human fibroblast, preferably isogenic to a subject that is in need of cell therapy, into a human oocyte which is then enucleated by known methods. The human fibroblast is optimally genetically modified to express GFP protein. The fibroblast and oocyte are fused by electrofusion as disclosed in earlier ACT and University of Massachusetts patent applications, incorporated by reference supra.

The NT embryo is activated substantially simultaneous to fusion.

The activated human NT embryo is cultured in a media suitable for maintaining human embryos until a gastrulating embryo is obtained which is 14 days old. At that point, the cells of the embryo are disaggregated and screened to identify cells that have become committed toward pancreatic lineage. This is effected by screening with monoclonal antibodies that specifically bind pancreatic markers.

These cells are separated from the other cells and placed in pharmaceutically acceptable buffered saline. These cells are then injected intravenously into a patient suffering from type I diabetes. The injected cells migrate to the pancreas and stably engraft therein. Successful engrafting is optimally determined by screening for the location and number of cells that express GFP. Efficacy is determined by monitoring the status of the patient, e.g., by monitoring changes in insulin levels after administration of cells. This procedure can be repeated if a suitable number of cells do not become stably engrafted.

### EXAMPLE 2

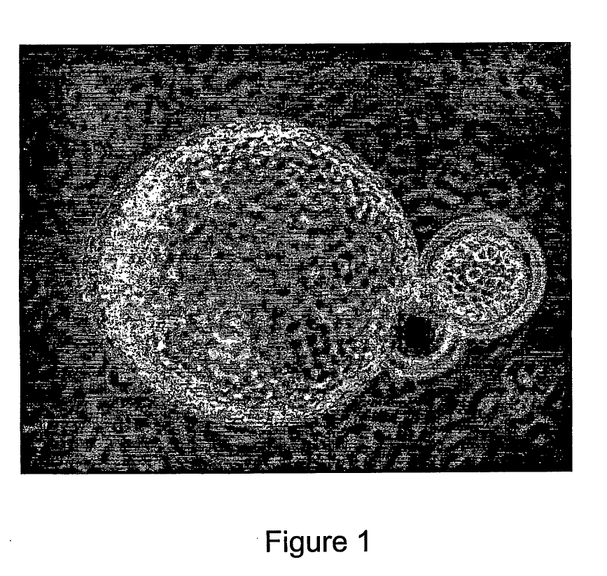

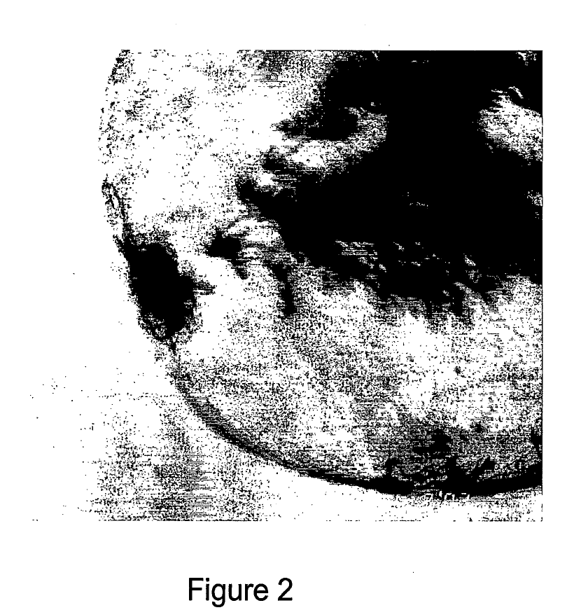

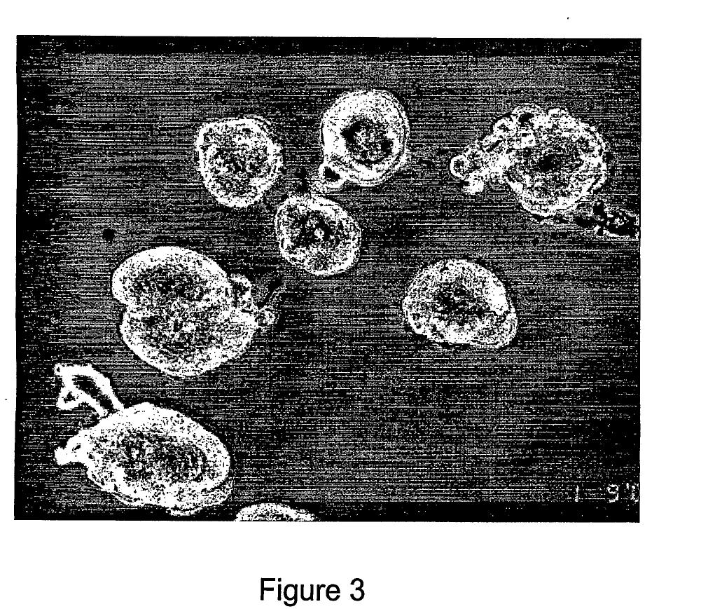

A rabbit embryo is produced by parthenogenesis. FIG. 1 shows a parthenogenetically activated rabbit blastocyst at day 8 (scale bar=100 microns). FIG. 2 shows a parthenogenetically activated rabbit blastocyst/embryonic sac cultured in vitro at day 22 (scale bar=500 microns). FIG. 3 shows embryonic cells isolated from parthenogenetically activated rabbit blastocyst/embryonic sac at day 22 (scale bar=50 microns).

### EXAMPLE 3

Although the goal of therapeutic cloning is to generate replacement cells and tissues that are genetically identical with those of the donor, numerous studies have shown that animals produced by somatic cell nuclear transfer inherit their mitochondria entirely or in part from the recipient oocyte and not from the donor cell⁶⁻⁸. This raises the question whether non-self mitochondrial proteins in cloned cells could lead to immunogenicity after transplantation and defeat the main objective of the procedure. For instance, it has been shown that mitochondrial peptides in mice are presented at the cell surface by non-classical major histocompatibility complex (MHC) class 1 molecules in combination with β2-microglobulin^{9,10}. It has also been shown that a single nonsynonymous nucleotide substitution in the mitochondrial ND1 gene results in a novel peptide that can be recognized by specific cytotoxic T cells¹¹. A similar situation occurs in rats, where a different nucleotide substitution in the ND1 gene results in a loss of histocompatibility¹². As mitochondrial peptides bound to class I molecules and displayed at the cell surface can serve as histocompatibility antigens in mice and rats, it is possible that similar systems are present in other mammalian species.

In this study, we tested the histocompatibility of nuclear transfer-generated cells and tissues in a large-animal model, the cow (Bos taurus). Cloned cardiac, skeletal muscle, and renal cell implants were not rejected and remained viable after being transplanted into the nuclear donor animal, even though they expressed a different mtDNA haplotype. Because the cloned cells were derived from early-stage fetuses, this approach is not an example of therapeutic cloning and would not be undertaken in humans.

We also investigated the use of nuclear transplantation to generate functional renal structures. It has been estimated that by 2010 more than two million patients will suffer from end-stage renal disease, at an aggregate cost of more than $1 trillion during the corning decade¹³. Because of its complex structure and function¹⁴, the kidney is one of the most challenging organs in the body to reconstruct. Previous efforts in kidney tissue engineering have been directed toward the development of an extracorporeal renal support system comprising both biologic and synthetic components¹⁵⁻¹⁷. This approach was first described by Aebischer et al.¹⁸⁻¹⁹ and is now being focused toward the treatment of acute rather than chronic renal failure. Humes et al.¹⁵ have shown that the combination of hemofiltration and a renal-assist device containing tubule cells can replace certain physiologic functions of the kidney when the filter and device are connected in an extravascular-perfusion circuit in uremic dogs. Heat exchangers, flow and pressure monitors, and multiple pumps are required for optimal functioning of this device^{20,21}. Although ex vivo organ substitution therapy would be life-sustaining, there would be obvious benefits for patients if such devices could be implanted on a long-term basis without the need for an extracorporeal-perfusion circuit or immunosuppressive drugs and/or immunomodulatory protocols. Synthetic, selectively permeable barriers can be used ex vivo to separate transplanted cells from the immune system of the body, but the implantation of such immunoisolation systems would pose considerable difficulties in both the long and short term^{22,25}.

Although nephrons have previously been grown in vitro from fetal and adult kidney cells in a number of mammalian species^{26, 27}, we show here in vivo reconstitution and structural remodeling of renal tissues from kidney cells. Renal cells from an early-stage cloned bovine fetus were used to generate functional immune-compatible renal tissues. The cloned renal cells were expanded in vitro, seeded onto renal units, and implanted back into the nuclear donor animal without immune destruction. The cells organized themselves into glomeruli- and tubule-like structures with the ability to excrete toxic metabolic waste products through a urinelike fluid.

Results and Discussion

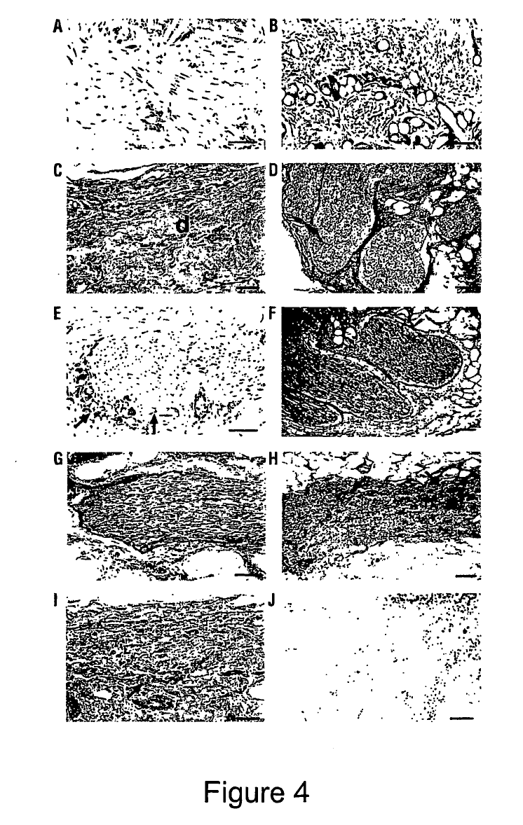

Cardiac and skeletal muscle constructs. Tissue-engineered constructs containing bovine cardiac (n=8) and skeletal muscle cells (n=8) were transplanted subcutaneously and retrieved six weeks after implantation. After retrieval of the first set of implants, a second set of constructs (n=12) from the same donor was transplanted for an additional 12 weeks. On a histologic level, the cloned cardiac tissue appeared intact and showed a well-organized cellular orientation with spindle-shaped nuclei (FIG. 4A). The retrieved tissue stained positively with troponin I antibodies, indicating the preservation of the cardiac muscle phenotype (FIG. 4B). The cloned skeletal cell explants showed spatially oriented tissue bundles with elongated multinuclear muscle fibers (FIGS. 4D, G). Immunohistochemical analysis using sarcomeric tropomyosin antibodies identified skeletal muscle fibers within the implanted constructs (FIG. 4F). In contrast to the cloned implants, the allogeneic control cell implants failed to form muscle bundles, and showed more inflammatory cells, fibrosis, and necrotic debris, consistent with acute rejection (FIGS. 4H, I).

Histologic examination revealed extensive vascularization throughout the implants, as well as the presence of multinucleated giant cells surrounding the remaining polymer fibers. Although nondegraded fibers were present in all tissue specimens, histomorphometric analysis of the explanted tissues indicated that the degree of immune reaction was significantly less in the cloned tissue sections than in the control (66±4 and 54±4 (mean+s.e.m.) total inflammatory cells/high-power field (HPF) for the cloned constructs at 6 weeks (first-set grafts) and 12 weeks (second-set grafts), respectively, vs. 93±3 and 80±3 cells/HPF for the constructs generated from the control cells, P<0.0005; FIG. 4F-G).

Immunocytochemical analysis using CD4- and CDS-specific antibodies identified approximately twofold-greater numbers of CD4⁺ and CD8⁺ T cells (13±1.3 and 14±1.4 cells/HPF, respectively, vs. 7±1.1 and 7±1.2 cells/HPF, P<0.00001) within the explanted first- and second-set control as compared with cloned constructs. Notably, cloned constructs from the first and second sets exhibited comparable levels of CD4 and CDS expression, arguing against the presence of an enhanced second-set reaction as would be expected if mtDNA-encoded minor antigen differences were present.

TABLE 1
Chemical analysis of fluid produced by renal units^{a}

| | Blood | Control 1 | Control 2 | Cloned |
|---|---|---|---|---|
| Sodium (mmol/l) | 141.7 ± 0.66 | 140.7 ± 0.67* | 141.3 + 0.67* | 133.2 ± 2.10* |
| Potassium (mmol/l) | 4.5 ± 0.03* | 7.4 ± 0.28 | 7.5 + 0.63 | 9.3 + 0.34* |
| Chloride (mmol/l) | 97.7 + 1.33* | 105.3 ± 0.33* | 105.5 + 0.21* | 79.3 ± 7.53* |
| Calcium (mg/dl) | 10.2 ± 0.06* | 6.6 ± 0.17 | 6.5 + 0.33 | 4.9 + 1.50* |
| Magnesium (mg/dl) | 2.6 ± 0.03* | 2.4 ± 0.05* | 2.5 + 0.12* | 0.9 ± 0.52* |

^{a}Mean ± s.e.m.
*P < 0.05 (comparison between blood, control, and cloned groups under the same conditions

Polyglycolic acid (PGA) is one of the most widely used synthetic polymers in tissue engineering^{28,29}. PGA polymers are biodegradable and biocompatible, and have been used in experimental and clinical settings for decades. Although the scaffolds are accepted by the immune system, PGA is known to stimulate a characteristic pattern of inflammation and ingrowth similar to that observed in the cloned constructs of the present study. However, this response, which is greatest at −12 weeks after implantation, can be considered as separate from the immune response to the transplanted cells, although there can clearly be interactions between the two³⁰⁻³⁵.

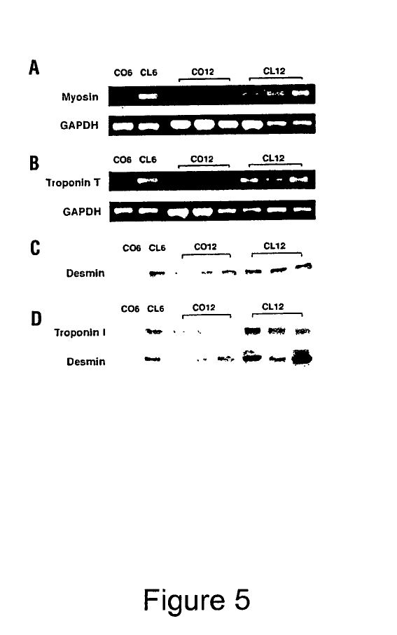

Semiquantitative RT-PCR and western blot analysis confirmed the expression of specific mRNA and proteins in the retrieved tissues despite the presence of allogeneic mitochondria. Mean expression intensities of myosin/GAPDH and troponin T/GAPDH in the cloned skeletal and cardiac implants were 0.22±0.03 and 0.15±0.02 (6 weeks) and 0.09±0.08 and 0.29±0.1 (12 weeks), respectively. In contrast, these expression intensities were significantly lower or absent in constructs generated from genetically unrelated cattle (0.02±0.01 and 0±0.00 at 6 weeks, P<0.005; and 0±0.01 and 0.02±0.1 at 12 weeks, P<0.05; FIGS. 5A, B). The cardiac and skeletal explants also expressed large amounts of desmin and troponin I proteins as determined by western blot analysis (FIGS. 5C, D). Desmin expression intensity was significantly greater in the cloned tissue sections than in the controls (85±1 and 68±4 vs. 30±2 and 16±2 at 6 weeks for the skeletal and cardiac implants, respectively, P<0.001; and 80±3 and 121±24 vs. intensities of troponin I in the cloned and control cardiac muscle explants were 68±4 and 16±2 at 6 weeks (P<0.001), respectively, and 94±7 and 54±12 at 12 weeks (P<0.05).

Western blot analysis of the first-set explants indicated an approximately sixfold greater expression intensity of CD4 in the control than in the cloned constructs at 6 weeks (30±10 and 32±3 for the control skeletal and cardiac implants, respectively, vs. 5±1 and 5±1 for the cloned skeletal and cardiac constructs, P<0.0005), confirming a primary immune response to the control grafts. The mean expression intensities of CDS were also significantly greater in the control than in the cloned constructs at 6 weeks (26±5 vs. 15±4, P<0.05). Twelve weeks after second-set implantation, mean expression intensities of CD4 and CDS remained significantly greater in the control than in the cloned constructs (23±4 vs. 12±3, respectively, for CD4, and 54±7 vs. 26±2, respectively, for CDS; P<0.005).

Renal constructs. Renal cells were isolated from a 56-day-old cloned metanephros and passaged until the desired number of cells were obtained. In vitro immunocytochemistry confirmed expression of renal-specific proteins, including synaptopodin (produced by podocytes), aquaporin-1 (AQP1, produced by proximal tubules and the descending limb of the loop of Henle), aquaporin-2 (AQP2, produced by collecting ducts), Tamm-Horsfall protein (produced by the ascending limb of the loop of Henle), and Factor VII (produced by endothelial cells). Cells expressing synaptopodin and AQP1 or AQP2 exhibited circular and linear patterns in two-dimensional culture, respectively. After expansion, the renal cells produced both erythro-poietin and 1,25-dihydroxyvitamin D₃, a key endocrinologic metabolite. The cloned cells produced 2.9+0.03 mlU/ml of erythro-poietin (compared with 0.0+0.03 mlU/ml for control fibroblasts (P<0.0005) and 2.9±0.39 mlU/ml for control renal cells) and were responsive to hypoxic stimulation (5.4±1.01 mlU/ml at 1% O2 vs. 2.9±0.03 mlU/ml at 20% O₂, P<0.02). The concentration of 1,25-dihydroxyvitamin D) was 20.2±1.12 pg/ml for the cloned cells, compared with <1 pg/ml for control fibroblasts (P<0.0002) and 18.6±1.72 pg/ml for control renal cells.

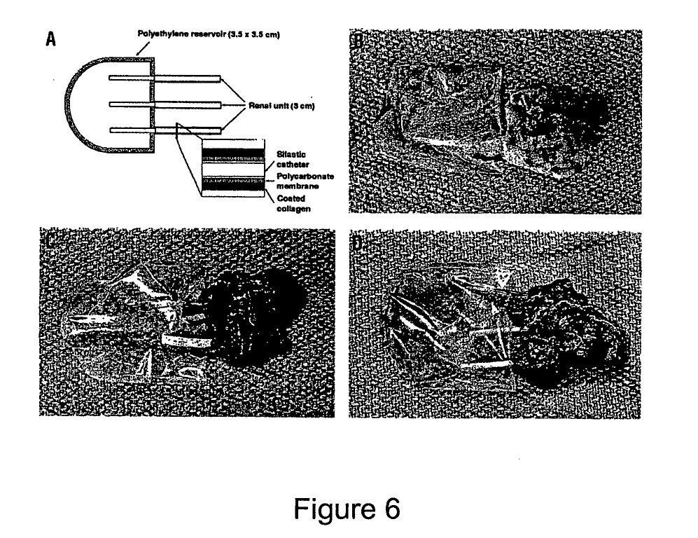

After expansion and characterization, the cloned cells were seeded onto collagen-coated cylindrical polycarbonate membranes. Renal devices with collecting systems were constructed by connecting the ends of three membranes with catheters that terminated in a reservoir (FIG. 6A). A total of 31 units (n=19 with cloned cells, n=6 without cells, and n=6 with cells from an allogeneic control fetus) were transplanted subcutaneously and retrieved 12 weeks after implantation into the nuclear donor animal.

On gross examination, the explanted units appeared intact, and straw-yellow fluid was seen in the reservoirs of the cloned group (FIG. 6D). The volume of fluid produced by the experimental group was sixfold greater than that produced by the control groups (0.60±0.04 ml vs. 0.10±0.01 ml and 0.13±0.04 ml in the allogeneic and unseeded control groups, respectively, P<0.00001). Chemical analysis of the fluid suggested unidirectional secretion and concentration of urea nitrogen (18.3+1.8 mg/dl urea nitrogen in the cloned group vs. 5.6±0.3 mg/dl and 5.0±0.01 mg/dl in the allogeneic and unseeded control groups, respectively, P<0.0005) and creatinine (2.5±0.18 mg/dl creatinine in the cloned group vs. 0.4±0.18 mg/dl and 0.4±0.08 mg/dl in the allogeneic and unseeded control groups, respectively, P<0.0005). Although the ratios of urine to plasma urea and creatinine were not physiologically normal, they were significantly greater than those of the controls, approaching up to 60% of what is considered to be within normal limits (the urine/plasma creatinine ratio was 6:1 in the cloned constructs vs. 10:1 in normal kidneys).

The physiologic function of the implanted units was further demonstrated by analysis of the electrolyte levels, specific gravity, and glucose concentrations of the collected fluid. The electrolyte levels in the fluid of the experimental group were significantly different from those of the plasma and the controls (Table 1), indicating that the implanted renal cells possessed filtration, reabsorption, and secretory functions. Urine specific gravity is an indicator of kidney function and reflects the action of the tubules and collecting ducts on the glomerular filtrate by giving an estimate of the solute concentration in the urine. The urine specific gravity of cattle is −1.025 and normally ranges from 1.020 to 1.040 (as compared with −1.010 in normal bovine serum)³⁶⁻³⁷. The specific gravity of the fluid produced by the cloned renal units was 1.027±0.001. The normal range of urine pH for adult herbivores is 7.0-9.0 (ref. 37). The pH of the fluid from the cloned renal units was 8.1±0.20. Glucose is reabsorbed in the proximal tubules and is seldom present in cattle urine. Glucose was undetectable (<10 mg/dl) in the cloned renal fluid (as compared with a blood glucose concentration of 76.6±0.04 mg/dl in the animals in the experimental group). The rate of excretion of minerals in cattle depends on a number of variables, including the mineral concentration in the animals' feed³⁶. However, the concentrations of magnesium and calcium, which are both reabsorbed in the proximal tubules and the loop of Henle, are normally <2.5 mg/dl and <5 mg/dl in bovine urine, respectively, and were 0.9±0.52 mg/dl and 4.9±1.5 mg/dl in the cloned urinelike fluid, respectively.

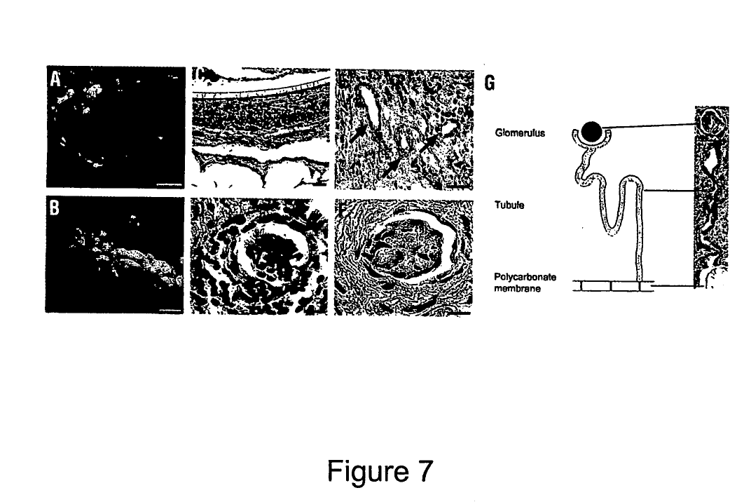

The retrieved implants showed extensive vascularization and had self-assembled into glomeruli and tubule-like structures (FIG. 7). The latter were lined with cuboid epithelial cells with large, spherical, pale-stained nuclei, whereas the glomeruli structures showed a variety of cell types with abundant red blood cells. There was a clear continuity between the mature glomeruli, their tubules, and the polycarbonate membrane (FIG. 7G). The renal tissues were integrally connected in a unidirectional manner to the reservoirs, resulting in the excretion of dilute urine into the collecting systems.

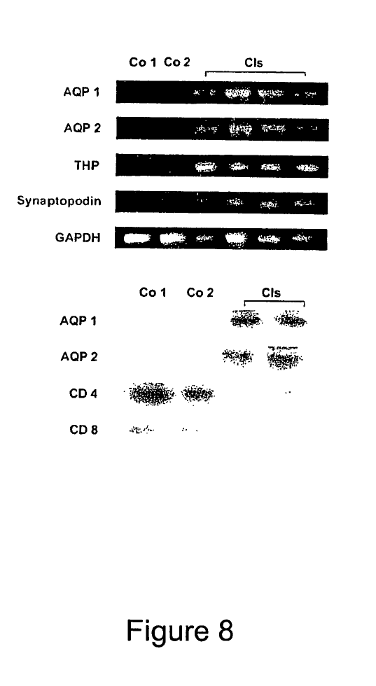

Immunohistochemical analysis confirmed the expression of renal-specific proteins, including AQP1, AQP2, synaptopodin, and Factor VIII (FIG. 7). Antibodies for AQP1, AQP2, and synaptopodin identified tubular, collecting-tubule, and glomerular segments within the constructs, respectively. In contrast, the allogeneic controls displayed a foreign-body reaction with necrosis, consistent with the finding of acute rejection. RT-PCR analysis confirmed the transcription of AQP1, AQP2, synaptopodin, and Tamm-Horsfall genes exclusively in the cloned group (FIG. 8). Cultured and cloned cells also expressed large amounts of AQP1, AQP2, synaptopodin, and Tamm-Horsfall protein as determined by western blot analysis. The expression intensities of CD4 and CDS, markers for inflammation and rejection, were also significantly higher in the control than in the cloned group (FIG. 8).

Mitochondrial DNA (mtDNA) Analysis

Previous studies showed that bovine clones harbor the oocyte mtDNA^{6-8, 38}. As discussed above, differences in mtDNA-encoded proteins expressed by cloned cells could stimulate a T-cell response specific for mtDNA-encoded minor histocompatibility antigens (miHAs)³⁹ when cloned cells are transplanted back to the original nuclear donor. The most straightforward approach to resolving the question of miHA involvement is the identification of potential antigens by nucleotide sequencing of the mtDNA genomes of the clone and the fibroblast nuclear donor. The contiguous segments of mtDNA that encode 13 mitochondrial proteins and tRNAs were amplified by PCR from total cell DNA in five overlapping segments for both donor-recipient combinations. These amplicons were directly sequenced on one strand with a panel of sequencing primers spaced at 500 bp intervals.

The resulting nucleotide sequences (13,210 bp) revealed nine nucleotide substitutions (Table 2) for the first donor-recipient combination (cardiac and skeletal constructs). One substitution was in the tRNA-Gly segment, and five substitutions were synonymous. The sixth substitution, in the ND1 gene, was heteroplasmic in the nuclear donor where one of the two alternative nucleotides was shared with the clone. A leucine or arginine would be translated at this position in ND1. The eighth and ninth substitutions resulted in amino acid interchanges of asparagine to serine and valine to alanine in the ATPase6 and ND4L genes, respectively. For the second donor-recipient combination (renal constructs), we obtained 12,785 bp from both the clone and the nuclear donor animal. The resulting sequences revealed six nucleotide substitutions (Table 2). One substitution was in the tRNA-Arg segment and three substitutions were synonymous. The fifth and sixth substitutions resulted in amino acid interchanges of isoleucine to threonine and threonine to isoleucine in the ND2 and ND5 genes, respectively.

TABLE 2
Nucleotide and amino acid substitutions distinguishing
nuclear donor and cloned cells

| Clone | | | | Amino acid |
|---|---|---|---|---|
| donor | Nuclear | Position^{a} | Gene | substitution |
| First combination | | | | |
| A | G | 13,080 | ND5 | — |
| T | C | 14,375 | ND6 | — |
| T | C | 7,851 | CoII | — |
| C | T | 8,346 | ATPase6 | — |
| A | G | 8,465 | ATPase6 | N→S |
| G | G/T | 3,501 | ND1 | R→ L/R |
| C | T | 9,780 | tRNA-Gly | |
| T | C | 10,432 | ND4L | V→A |
| G | A | 11,476 | ND4 | — |
| Second combination | | | | |
| T | C | 4,945 | ND2 | I→T |
| C | T | 7,580 | CoII | — |
| A | G | 9,095 | CoIII | — |
| C | T | 10,232 | tRNA-Arg | — |
| G | A | 10,576 | ND4 | — |
| C | T | 12,377 | ND5 | T→I |

^{a}Position in GenBank, accession no. J013494.

The identification of two amino acid substitutions that distinguish the clone and the nuclear donor confirms that a maximum of only two miHA peptides could be defined for each donor-recipient combination. Given the lack of knowledge about peptide-binding motifs for bovine MHC class I molecules, there is no reliable method to predict the impact of these amino acid substitutions on the ability of mtDNA-encoded peptides either to bind to bovine class I molecules or to activate CD8⁺ cytotoxic T lymphocytes (CTLs).

Despite the potential immunogenicity of the two amino acid substitutions in the first donor-recipient combination, it was clear that the cloned devices functionally survived for the duration of the experiments without significant increases in infiltration of second-set devices by CD4⁺ and CD8⁺ T lymphocytes. Specifically, cloned cardiac and skeletal tissues remained viable for more than three months after second-set transplantation (comparable to in vitro control specimens). Multiple, viable, myosin- and troponin I-containing cells were observed throughout the tissue constructs, consistent with functionally active protein synthesis and expression. This direct assessment of graft function does not provide any evidence to support the activation of a T-cell response to cloned tissue-specific histocompatibility antigens in this donor-recipient combination.

These findings are consistent with those of the second transplant donor-recipient combination. The cloned renal cells derived their nuclear genome from the original fibroblast donor and their mtDNA from the original recipient oocyte. A relatively limited number of mtDNA polymorphisms have been shown to define maternally transmitted miHAs in mice³⁹. This class of miHAs stimulates both skin allograft rejection in vivo and expansion of CTLs in vitro³⁹, and might constitute a barrier to successful clinical use of such cloned devices, as has been hypothesized in chronic rejection of MHC-matched human renal transplants^{40,41}. We chose to investigate a possible anti-miHA T-cell response to the cloned renal devices through both DTH testing in vivo and Elispot analysis of IFN**-secreting T cells in vitro. An in vivo assay of anti-miHA immunity was chosen on the basis of the ability of skin allograft rejection to detect a wide range of miHAs in mice with survival times exceeding ten weeks⁴² and the relative insensitivity of in vitro assays in detecting miHA incompatibility, highlighted by the requirement for in vivo priming to generate CTLs⁴³. Using DTH testing in vivo, we did not see an immunological response directed against the cloned cells. Cloned and control allogeneic cells were intradermally injected back into the nuclear donor animal 80 days after the initial transplantation. A positive DTH response was observed after 48 h for the allogeneic control cells but not for the cloned cells (diameter of erythema and induration of about 9×4.5 mm, 12×10 mm, and 11×11 mm vs. 0, 0, and 0 mm, respectively, P<0.02).

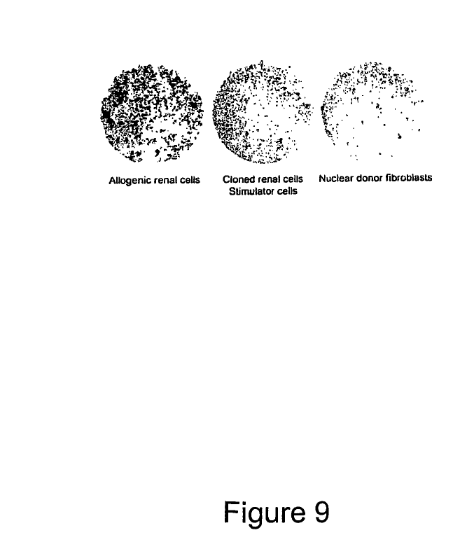

The results of DTH analysis were mirrored by Elispot-derived estimates of the frequencies of T cells that secreted IFNγ after in vitro stimulation. Primary B lymphocytes were harvested from the transplanted recipient one month after retrieval of the devices. These primary B lymphocytes were stimulated in primary nixed-lymphocyte cultures with allogeneic renal cells, cloned renal cells, and nuclear donor fibroblasts (FIG. 9). Surviving T cells were restimulated in anti-IFNγ-coated wells with either nuclear donor fibroblasts (autologous control) or the respective stimulators used in the primary mixed-lymphocyte cultures. Elispot analysis revealed a relatively strong T-cell response to allogeneic renal stimulator cells relative to the responses to either cloned renal cells or nuclear donor fibroblasts. A mean of 342 spots (s.e.m.±36.7) was calculated for allogeneic renal cell-specific T cells. Significantly lower numbers of IFNγ-secreting T cells responded to cloned renal cells and nuclear donor fibroblasts. Nuclear donor fibroblast-stimulated T cells yielded 45 (s.e.m.±1.4) and 55 (s.e.m.±5.7) spots after secondary stimulation with cloned renal and nuclear donor fibroblast stimulators, respectively. Likewise, cloned renal cell-stimulated T cells yielded 61 (s.e.m.±2.8) and 33.5 (s.e.m.±0.7) spots with the same stimulator populations. These results corroborate both the relative CD4 and CD8 expression in western blots (FIG. 5), and the results of in vivo DTH testing, supporting the conclusion that no detectable rejection response specific for cloned renal cells occurred after either primary or secondary challenge.

### CONCLUSIONS

Our results suggest that cloned cells and tissues with allogeneic mtDNA can be grafted back into the nuclear donor organism without destruction by the immune system, although further studies will be necessary to rule out the possibility of immune rejection with other donor-recipient transplant combinations. It is important to note that bovine ES cells capable of differentiating into specified tissue in vitro have not yet been isolated. It was therefore necessary in the present study to generate an early-stage bovine embryo. This strategy could not be applied in humans, as ethical considerations require that preimplantation embryos not be developed in vitro beyond the blastocyst stage⁴⁴⁻⁴⁶. However, human and primate ES cells have been successfully differentiated in vitro into derivatives of all three germ layers, including beating cardiac muscle cells, smooth muscle, and insulin-producing cells, among others⁴⁷⁻⁵².

Although functional tissues can be engineered using adult native cells^{53,54}, the ability to bioengineer primordial stem cells into more complex functional structures such as kidneys would overcome the two major problems in transplantation medicine: immune rejection and organ shortage. It is clear that a staged developmental strategy will be required to achieve this ultimate goal. The results presented here suggest that nuclear transplantation may overcome the hurdle of immune incompatibility.

Experimental Protocol

Adult bovine cell line derivation. Dermal fibroblasts were isolated from adult Holstein steers by ear notch. Tissue samples were minced and cultured in DMEM (Gibco, Grand Island, N.Y.) supplemented with 15% FCS (HyClone, Logan, Utah), L-glutamine (2 mM), nonessential amino acids (100 μM) p-mercaptoethanol (154 μM), and antibiotics at 38° C. in a humidified atmosphere of 5% CO₂ and 95% air. The tissue explants were maintained in culture and a fibroblast cell monolayer established. The cell strain was maintained in culture, passaged, cryopreserved in 10% dimethyl sulfoxide, and stored in liquid nitrogen before nuclear transfer. Experimental protocols followed guidelines approved by the Children's Hospital (Boston, Mass.) and Advanced Cell Technology (Worcester, Mass.) Institution Animal Care and Use Committees.

Nuclear transfer and embryo culture. Bovine oocytes were obtained from abattoir-derived ovaries as described elsewhere³⁸. Oocytes were mechanically enucleated at 18-22 h post maturation, and complete enucleation of the metaphase plate was confirmed with bisbenzimide (Hoechst 33342; Sigma, St. Louis, Mo.) dye under fluorescence microscopy. A suspension of actively dividing cells was prepared immediately before nuclear transfer. Single donor cells were selected and transferred into the perivitelline space of the enucleated oocytes. Fusion of the cell-oocyte complexes was accomplished by applying a single pulse of 2.4 kV/cm for 15 μs. Nuclear transfer embryos were activated as described elsewhere by Presicce et al.⁵⁵ with slight modifications. Briefly, reconstructed embryos were exposed to 5 μM ionomycin (CalBiochem, La Jolla, Calif.) in Tyrode lactate-HEPES for 5 min at room temperature followed by a 6 h incubation with 5 μg/ml cytochalasin B (Sigma) and 10 μl/ml cycloheximide (Sigma) in astroglial cell-culture medium. The resulting blastocysts were nonsurgically transferred into progestin-synchronized recipients.

Cell culture and seeding. Cardiac and skeletal tissue from five- to six-week-old cloned and natural fetuses were retrieved. The cells were isolated by the explant technique and cultured using DMEM as above. Both muscle cell types were expanded separately until desired numbers of cells were obtained. The cells were trypsinized, washed, and seeded in 1×2 cm PGA polymer scaffolds with 5×10⁷ cells. Vials of frozen donor cells were thawed and passaged before seeding the second-set scaffolds. Renal cells were derived from seven-to eight-week-old cloned and natural fetuses. Metanephros were surgically dissected under a microscope, and cells were isolated by enzymatic digestion using 0.1% (wt/vol) collagenase/dispase (Roche, Indianapolis, Ind.) and cultured using DMEM supplemented as above. Cells were passed by 1:3 or 1:4 every three to four days, and expanded until desired cell numbers (˜6×10⁸) were obtained. The cells were seeded in coated collagen with 2×10⁷ cells/cm² density. Vials of frozen donor cells were thawed and passaged for DTH testing and for use in the in vitro proliferative assays.

Polymers and renal devices. Unwoven sheets of polyglycolic acid polymers (1 cm×2 cm×3 mm) were used as cell delivery vehicles (Albany International, Mansfield, Mass.). The polymer meshes were composed of fibers 15 |μm in diameter with an interfiber distance of 0-200 μm with 95% porosity. The scaffold was designed to degrade by hydrolysis in 8-12 weeks. Renal devices with collecting systems were constructed by connecting the ends of three cylindrical polycarbonate membranes (3 cm long, 10 μm thick, 2 μm pore size, 1.4 mm internal diameter; Nucleopore Filtration Products, Cambridge, Mass.) with 16 G Silastic catheters that terminated in a 2 ml reservoir made from polyethylene sealed along the edge by the application of pressure and heat. The distal end of the cylindrical membranes was also sealed, and the membranes coated with type 1 collagen (0.2 cm thickness) extracted from rat-tail collagen.

Implantation and analysis of fluid. The cell-polymer constructs were implanted into the flank subcutaneous tissue of the same steer from which the cells were cloned. Fourteen constructs (eight first-set and six second-set) for each cell type were implanted. Control group constructs, with cells isolated from an allogeneic fetus, were implanted on the contralateral side. The implanted constructs were retrieved at 6 weeks (first set) and 12 weeks (second set) after implantation. The renal units were also derived from a single fetus. Thirty-one units (n=19 with cloned cells, n=6 without cells, and n=6 with cells isolated from an allogeneic, age-matched control fetus) were transplanted subcutaneously and retrieved 12 weeks after implantation. The solute concentrations of urea nitrogen, creatinine, and electrolytes were measured in the accumulated fluid in the explanted renal reservoirs using standard techniques.

DTH testing. Cloned, allogeneic, and autologous cells were intradermally injected into the nuclear donor animal (1×10⁶ cells in 0.1 ml in triplicate). Three sites were chosen for softest skin: the left and right side of the tail, and just below the anus. After each site was shaved and prepared, the cells were injected in a row about 2 cm apart. The area of erythema and induration was measured (blinded) after 24-72 h, with 48 h being considered the optimal time to detect a DTH response.

Elispot analysis. Bovine recipient peripheral blood lymphocytes (PBLs) were isolated from whole blood and cultured for six days with irradiated allogeneic renal cells, cloned renal cells, and nuclear donor fibroblasts at 37° C. in RPMI medium plus 10% FCS and human interleukin-2 (20 units/ml) (Chiron, Emeryville, Calif.). On day 6, the stimulated PBLs were harvested and plated at 25,000 cells/well in duplicate wells of a 96-well Multiscreen plate, which had been coated overnight with mouse anti-bovine IFN-γ (10 (1 g/ml) (Biosource, Camarillo, Calif.). A total of 50,000 cells matched to the primary culture stimulators were added to the respective wells. The plate was incubated for 24 h at 37° C. and washed 3× with 0.5% Tween-20 and 4× in distilled water. Biotinylated mouse anti-bovine IFNy (5 (Ig/ml) (Biosource) was added, and the plate was incubated for 2 h at 37° C. The plate was washed as above and alkaline phos-phatase-conjugated anti-biotin (1:1000 dilution; Vector, Burlingame, Calif.) was added and incubated for 1 h at room temperature. The plate was washed and 100 μl of 5-bromo-4-chloro-3-indolyl phosphate/nitro blue tetrazolium (BC/NBT) (Sigma) was added for development of spots. After development, BCIP/NBT was washed out of the wells with distilled water. The wells were photographed and analyzed with Immunospot software (Cellular Technologies, Cleveland, Ohio).

Histological and immunohistochemical analyses. Sections (5 μm) of 10% (wt/vol) buffered formalin-fixed paraffin-embedded tissue were cut and stained with hematoxylin and eosin (H&E). Immunohistochemical analyses were done with specific antibodies to identify the cell types in retrieved tissues with cryostat and paraffin sections. Monoclonal sarcomeric tropomyosin (Sigma) and troponin I (Chemicon, Temecula, Calif.) antibodies were used to detect skeletal and cardiac fibers, respectively. Monoclonal synaptopodin (Research Diagnostics, Flanders, N.J.), polyclonal AQP1 and AQP2, and polyclonal Tamm-Horsfall protein (Biomedical Technologies, Stoughton, Mass.) were used to detect glomerular and tubular tissue, respectively. Monoclonal CD4 and CD8 (Serotec, Raleigh, N.C.) antibodies were used to identify T cells for immune rejection. Specimens were routinely processed for immunostaining. Pretreatment for high-temperature antigen unmasking pretreatment with 0.1% trypsin was conducted using a commercially available kit according to the manufacturer's recommendations (T-8128; Sigma). Antigen-specific primary antibodies were applied to the deparaffinized and hydrated tissue sections. Negative controls were treated with nonimune serum instead of the primary antibody. Positive controls consisted of normal tissue. After washing with PBS, the tissue sections were incubated with a biotinylated secondary antibody and washed again. A peroxidase reagent (diaminobenzidine) was added. Upon substrate addition, the sites of antibody deposition were visualized by a brown precipitate. Counterstaining was performed with Gill's hematoxylin. To determine the degree of immunoreaction, the immune cells were counted under five high-power fields per section (HPF, x200) using computerized histomorphometrics (BioImaging Analyses Software, NIH Image 6.2, NIH, Rockville, Md.).

Erythropoietin and 1,25-dihydroxyvitamin D₃ assays. Cloned renal cells, allogeneic renal cells, and cloned fibroblasts were grown to confluence in 60 mm culture dishes (in quadruplicate) at 20% O₂, 5% CO₂. After washing 3×, the cells were incubated in either serum-free medium for 24 h (erythropoietin) or serum-free medium with 1,25-hydroxyvitamin D₃ (1 ng/ml) for 12 h. Erythropoietin production in the supernatants was measured by the double-antibody sandwich enzyme-linked immunosorbent assay (ELISA) using a Quantikine IVD Erythropoietin ELISA kit (R&D Systems, Minneapolis, Minn.). Erythropoietin production was also measured in the supernatant of cells that were incubated in a hypoxic chamber (1% O₂, 5% CO₂) for 4 h. Production of 1,25-dihydroxyvitamin D₃ in the supernatants was measured by radioimmiunoassay using a ¹²⁵I RIA kit (DiaSorin, Stillwater, Minn.).

Mitochondrial DNA analyses. Mitochondrial DNA products ranging in size from 3 kb to 3.8 kb were amplified by PCR using Advantage-GC Genomic Polymerase (Clontech, Palo Alto, Calif.) and total genomic DNA templates from the clone and nuclear donor. The regions of the mitochondria that were amplified included all of the protein-coding sequences and the intervening tRNAs. PCR products were electrophoresed in 1% (wt/vol) SeaPlaque GTG agarose (Rockland, Me.), extracted from the gels with the use of QIAquick Gel Extraction Kits (Qiagen, Valencia, Calif.), and sequenced by the Molecular Biology Core Facility (Mayo Clinic, Rochester, Minn.) with a series of primers located at ˜500-base intervals.

RNA isolation and cDNA synthesis. Freshly retrieved tissue implants were harvested and frozen immediately in liquid nitrogen. The tissue was homogenized in RNAzol reagent (Tel-Test, Friendswood, Tex.) at 4° C. using a tissue homogenizer. RNA was isolated according to the manufacturer's protocol (Tel-Test). Complementary DNA was synthesized from 2 μg RNA using the Superscript II reverse transcriptase (Gibco) and random hexamers as primers.

PCR. For PCR amplification, 1 ml of cDNA with 1 unit Taq DNA polymerase (Roche), 200 mM DNTP, and 10 pM of each primer were used in a final volume of 30 ml. Myosin for skeletal muscle tissue was amplified from cDNA with primers 5′-TGAATTCAAGGAGGCGTTTCT-3′ and 5′-CAGGGCTTCCACTTCTTCTTC-3′. Troponin T for cardiac tissue was done with primers 5′-AAGCGCATGGAGAAGGACCTC-3′ and 5′-GGATGTAGCCGCCGAAGTG-3′. Synaptopodin for glomerulus was amplified from cDNA with primers 5′-GGTGGCCAGTGAGGAGGAA-3′ and 5′-TGCTCGCCCAGA-CATCTCTT-3′. Podocalyxin for glomerulus was done with primers 5′-CTCTCGGCGCTGCTGCTACT-3′ and 5′-CGCTGCTGGTCCTCCTCTG-3′. AQP1 for tubule was done with primers 5′-CAGCATGGCCAGCGACGAGTTCAAGA-3′ and 5′-TGTCGTCGGCATCCAGGTCATAC-3′; AQP2 for tubule was done with primers 5′-GCAGCATGTGGGARCTNM-3′ and 5′-CTYACIGCRTTIACNGCNAGRTC-3′. Tamm-Horsfall protein for tubule was done with primers 5′-AACTGCTCCGCCACCAA-3′ and 5′-CTCACAGTGCCTTCCGTCTC-3′. PCR products were visualized with agarose gel electrophoresis and ethidium bromide staining.

Western blot analysis. Tissue was homogenized in lysis buffer using a tissue homogenizer. After measuring protein concentration (Bio-Rad), equal protein amounts were loaded on 10% SDS-PAGE. Proteins were blotted onto polyvinylidene fluoride membranes, which were incubated with primary antibodies for 1 h at room temperature. Desmin (Santa Cruz Biotech, Santa Cruz, Calif.) antibodies were used to detect skeletal tissue; desmin and troponin I (Santa Cruz Biotech) antibodies were used to detect cardiac tissue; and synaptopodin, AQP1, AQP2, and Tamm-Horsfall protein (Research Diagnostics, Flanders, N.J.) were used to detect glomerular and tubular tissue, respectively. Monoclonal CD4 and CD8 antibodies were used as markers for inflammation and rejection. Subsequently, membranes were incubated with secondary antibodies for 30 min. The signal was visualized using the ECL system (NEN, Boston, Mass.).

Statistical analysis. Data are presented as mean±s.e.m. and compared using the two-tailed Student's t-test. Differences were considered significant at P<0.05.

While the invention has been described with respect to certain specific embodiments, it will be appreciated that many modifications and changes thereof may be made by those skilled in the art without departing from the spirit of the invention. It is intended, therefore, by the appended claims to cover all modifications and changes that fall within the true spirit and scope of the invention.

### REFERENCES

1. Lanza, R. P. ef al. The ethical reasons for stem cell research. Science 293, 1299 (2001).
2. Atala, A. & Lanza, R. P. Methods of Tissue Engineering (Academic Press, San Diego, Calif., 2001).
3. Atala, A. & Mooney, D. Synthetic Biodegradable Polymer Scaffolds (Birkhauser, Boston, Mass., 1997).
4. Machluf, M. & Atala, A. Emerging concepts for tissue and organ transplantation. Graft 1, 31-37 (1998).
5. Lanza, R. P, Cibelli, J. B. & West, M. D. Prospects for the use of nuclear transfer inhuman transplantation. Nat. Biotechnol. 17,1171-1174(1999).
6. Evans, M. J. era/. Mitochondrial DNA genotypes in nuclear transfer-derived cloned sheep. Nat. Genet. 23, 90-93 (1999).
7. Hiendleder, S., Schmutz, S. M., Erhardt, G., Green, R. D. & Plante, Y. Transmitochondrial differences and varying levels of heteroplasmy in nuclear transfer cloned cattle. Mol. Reprod. Dev. 54, 24-31 (1999).
8. Steinborn, R. ef al. Mitochondrial DNA heteroplasmy in cloned cattle produced by fetal and adult cell cloning. Nat. Genet. 25, 255-257 (2000).
9. Vyas, J. M. et al. Biochemical specificity of H-2M3a: stereospecificity and space-filling requirement at position 1 maintains N-formyl peptide binding. J. Immunol. 149,3605-3611 (1992).
10. Morse, M. et al. The COI mitochondrial gene encodes a minor histocompatibility antigen presented by H2-M3. J. Immunol. 156, 3301-3307 (1996).
11. Loveland, B., Wang, C. R., Yonekawa, H., Hermel, E. & Lindahl, K. R Maternally transmitted histocompatibility antigens of mice: a hydrophobic peptide of a mitochondrial encoded protein. Ce//60, 971-980 (1990).
12. Davies, J. D. et al. Generation of T cells with lytic specificity for atypical antigens. I. A mitochondrial antigen in the rat. J. Exp. Med. 173, 823-832 (1991).
13. Lysaght, M. J. Maintenance dialysis population dynamics: current trends and long-term implications. J. Am. Soc. Nephrol. 13, S37-S40 (2002).
14. Amiel, G. E. & Atala, A. Current and future modalities for functional renal replacement. Urol. Clin. 26, 235-246 (1999).
15. Humes, H. D., Buffington, D. A., MacKay, S. M., Funke, A. J. & Weitzel, W. F. Replacement of renal function in uremic animals with a tissue-engineered kidney. Nat. Biotechnol. 17, 451-455 (1999).
16. Cieslinski, D. A. & Humes, H. D. Tissue engineering of a bioartificial kidney. Biotechnol. Bioeng. 43, 781-791 (1994).
17. MacKay, S. M., Kunke, A. J., Buffington, D. A. & Humes, H. D. Tissue engineering of a bioartificial renal tubule. ASAIO J. 44,179-183 (1998).
18. Aebischer, P., Ip, T. K, Panel, G. & Galletti, P. M. The bioartificial kidney: progress towards an ultrafiltration device with renal epithelial cells processing. Life Support Syst. 5, 159-168 (1987).
19. Ip, T., Aebischer, P. & Galletti, P. M. Cellular control of membrane permeability. Implications for a bioartificial renal tubule. ASAIO Trans. 34, 351-355 (1988).
20. Humes, H. D. Renal replacement devices, in Principles of Tissue Engineering; Edn. 2 (eds Lanza, R. P., Langer, R. & Vacanti, J.) 645-653 (Academic Press, San Diego, 2000).
21. Amiel, A., Yoo, J. & Atala, A. Renal therapy using tissue engineered constructs and gene delivery. World J. Urol. 18, 71-79 (2000).
22. Lanza, R. P, Hayes, J. L. & Chick, W. L. Encapsulated cell technology. Nat. Biotechnol. 14, 1107-1111 (1996).
23. Kuhtreiber, W. M., Lanza, R. P. & Chick, W. L. (eds). Cell Encapsulation Technology and Therapeutics (Birkhauser, Boston, 1998).
24. Lanza, R. P & Chick, W. L. (eds). Immunoisolation of Pancreatic Islets (R. G. Landes, Austin, Tex. 1994).
25. Joki, T. ef al. Continuous release of endostatin from microencapsulated engineered cells for tumor therapy. Nat. Biotechnol. 19, 35-39 (2001).
26. Qiao, J., Sakurai, H. & Nigam, S. K. Branching morphogenesis independent of mesenchymal-epithelial contact in the developing kidney. Proc. Natl. Acad. Sci. USA 96, 7330-7335 (1999).
27. Humes, H. D., Krauss, J. C., Cieslinski, D. A. & Funke, A. J. Tubulogenesis from isolated single cells of adult mammalian kidney: clonal analysis with a recombinant retrovirus. Am. J. Phys/ol. 271, F42-F49 (1996).
28. Lanza, R. P., Langer, R & Vacanti, J. Principles of Tissue Engineering (Academic Press, San Diego, Calif., 2000).
29. Atala, A. Future perspectives in reconstructive surgery using tissue engineering. Urol. Clin. 26, 157-166 (1999).
30. Santavirta, S. ef al. Immune response to polyglycolic acid implants. J. Bone Joint Surg. Br. 72, 597-600(1990).
31. Paivarinta, U. ef al. Intraosseous cellular response to biodegradable fracture fixation screws made of polyglycolide or polylactide. Arch. Orthop. Trauma Surg. 112, 71-74(1993)
32. Bostman, O. M. & Pihlajamaki, H. K. Adverse tissue reactions to bioabsorbable fixation devices. Clin. Orthop. 371, 216-227 (2000).
33. Ruuskanen, M. et al. Evaluation of self-reinforced polyglycolide membrane implanted in the subcutis of rabbits. Ann. Chir. Gynaecol. 88, 308-312 (1999).
34. Weiler, A., Helling, H. J., Kirch, U., Zirbes. T. K & Rehm, K. E. Foreign-body fracture fixation: experimental study in sheep. J. Bone Joint Surg. Br. 78, 369-376 (1996).
35. Pariente, J. L., Kim, B. S. & Atala, A. In vitro compatibility assessment of naturally-derived and synthetic biomaterials using normal human urothelial cells. J. Biomed. Mat. Res. 55, 33-39 (2001).
36. Rosenberger, G. Clinical Examination of Cattle (Verlag Paul Parey, Berlin, 1979), pp. 275-281.
37. Smith, B. P. Large Animal Internal Medicine: Diseases of Horses, Cattle, Sheep and Goats, Edn. 2 pp. 467-469 (Mosby, St. Louis, 1996).
38. Lanza, R. P. ef al. Cloning of an endangered species (Bos gaurus) using inter-species nuclear transfer. Cloning Z, 79-90 (2000).
39. Fischer Lindahl, K., Hermel, B., Loveland, B. E. & Wang, C. R. Maternally transmitted antigen of mice. Ann. Rev. Immunol. 9, 351-372 (1991).
40. Hadley, G. A., Linders, B. & Mohanakumar, T, Immunogenicity of MHC class I alloantigens expressed on parenchymal cells in the human kidney. Transplantation 54, 537-542 (1992).
41. Yard, B. A. ef al. Analysis of T cell lines from rejecting renal allografts. Kidney Int. 43,3133-3138(1993).
42. Bailey, D. W. Genetics of histocompatibility in mice. I. New loci and congenic lines. Immunogenetics Z, 249-256 (1975).
43. Mohanakumar, T. The Role of MHC and Non-MHC Antigens in Allograft Immunity pp. 1-115 (R. G. Landes Company, Austin, Tex., 1994).
44. Lanza, R. P, Cibelli, J. B. & West, M. D. Human therapeutic cloning. Nat. Med. 5, 975-977(1999).
45. Cibelli, J. B. ef al. Somatic cell nuclear transfer in humans: pronuclear and early embryonic development. e-biomed: J. Regen. Med. 2, 25-31 (2001).
46. Lanza, R. P. ef al. The ethical validity of using nuclear transfer in human transplantation. JAMA 284, 3175-3179 (2000).
47. Itskovitz-Eldor, J. ef al. Differentiation of human embryonic stem cells into embryoid bodies comprising the three embryonic germ layers. Mol. Med. 5, 88-95 (2000).
48. Schuldiner, M., Yanuka, O., Itskovitz-Eldor, J., Melton, D. A. & Benvenisty, N. Effects of eight growth factors on the differentiation of cells derived from human embryonic stem cells. Proc. Natl. Acad. Sci USA 97, 11307-11312 (2000).
49. Kaufman, D. S. et al. Directed differentiation of human embryonic stem cells into hematopoietic colony forming cells. Blood 94 (Suppl. 1, part 1 of 2), 34a (1999).
50. Reubinoff, B. E. ef al. Neural progenitors from human embryonic stem cells. Nat. Biotechnol 19, 1134-1140(2001).
51. Reubinoff, B. E., Pera, M. F, Fong, C. Y., Trounson, A. & Bongso, A. Embryonic stem cell lines from human blastocysts: somatic differentiation in vitro. Nat. Biotechnol. 18, 399-404 (2000).
52. Cibelli, J. B. et al Parthenogenetic stem cells in nonhuman primates. Science 295, 819(2002).
53. Oberpenning, F. O., Meng, J., Yoo, J. & Atala, A. De novo reconstitution of a functional urinary bladder by tissue engineering. Nat. Biotechnol. 17,149-155 (1999).
54. Kaushal, S. et al. Circulating endothelial cells for tissue engineering of small diameter vessels. Nat. Med. 7,1035-1040 (2001).
55. Presicce, G. A. & Yang, X. Parthenogenetic development of bovine oocytes matured in vitro for 24 hr and activated by ethanol and cycloheximide. Mol. Reprod. Dev. 38, 380-385 (1994).

What is claimed is:

1. A method of cell therapy which comprises:
(i) obtaining a nuclear transfer (NT) embryo;
(ii) allowing said NT embryo to develop into a gastrulating embryo that ranges from about one cell to six weeks in age:
(iii) isolating a cell or cells from said embryo; and
(iv) introducing said cell or cells into a subject that is in need of cell therapy.

2. The method of claim 1 wherein the NT embryo ranges in age from 2 weeks to 4 weeks.

3. The method of claim 1 wherein the cells have commenced becoming committed to a specific lineage.

4. The method of claim 1 wherein said cells are selected from the group consisting of myocardiocytes, pancreatic cells, hemagioblasts, hematopoietic progenitors. CNS progenitors and hepatocytes.

5. The method of claim 1 wherein the. cell therapy is used to treat a defect selected from the group consisting of a cardiac defect, lung disorder, immune cell deficiency. neural disorder, liver disorder, autoimmune disease, age-related disorder. cancer, proliferative disorder, allergic disorder, and blood related disorder.

6. The method of claim 1 wherein said cells are committed to a desired cell lineage.

7. The method of claim 6 wherein said cells express at least one marker characteristic of a particular cell lineage.

8. The method of claim 1 wherein said subject has cancer.

9. The method of claim 1 wherein the subject has an autoimmune disorder.

10. The method of claim 1 wherein the subject has a neural disorder.

11. The method of claim 1 wherein said subject has ALS, Parkinson's disease, Huntington's disease, Alzheimer's disease, or myasthenia gravis.

12. The method of claim 1 wherein the NT embryo is produced using a somatic cell that is genetically modified.

13. A method of cell therapy which comprises:
(i) obtaining a mammalian embryo made up of cells that are histocompatible with a mammalian individual that is in need of cell transplant therapy;
(ii) allowing said embryo to develop into a gastrulating embryo;
(iii) isolating a cell or cells from said embryo; and
(iv) introducing said cell or cells into said individual in need of cell therapy.

14. The method of claim 13 wherein the embryo is an NT embryo.

15. The method of claim 13 wherein the embryo is an NT embryo that is genetically modified so that it is incapable of developing into a viable mammal.

16. The method of claim 13, wherein the embryo is an NT embryo wherein the donor cell and the oocyte are from different species.

17. The method of claim 16, wherein the donor cell is a human cell.

18. The method of claim 19, wherein the oocyte is from a mammal selected from the group consisting of rabbit, bovine, and non-human primate.

19. The method of claim 13, wherein the embryo is an androgenetic embryo.

20. The method of claim 19, wherein the embryo is a haploid androgenetic embryo.

21. The method of claim 19, wherein the embryo is a diploid androgenetic embryo.

22. The method of claim 13 wherein the cells isolated from the have commenced becoming committed to a specific lineage.

23. The method of claim 13 wherein the cell or cells are isolated from a gastrulating embryo that ranges from about one cell to six weeks in age:

24. The method of claim 13 wherein the cell or cells are isolated from a gastrulating embryo that ranges in age from 2 weeks to 4 weeks.

25. The method of claim 13 wherein said cells are selected from the group consisting of myocardiocytes, pancreatic cells, hemagioblasts, hematopoietic progenitors. CNS progenitors and hepatocytes.

26. The method of claim 13 wherein the. cell therapy is used to treat a defect selected from the group consisting of a cardiac defect, lung disorder, immune cell deficiency. neural disorder, liver disorder, autoimmune disease, age-related disorder. cancer, proliferative disorder, allergic disorder, and blood related disorder.

27. The method of claim 13 wherein the embryo is produced using a somatic cell that is genetically modified.
